(19) United States
(12) Patent Application Publication
(10) Pub. No.: US 2026/0069599 A1
(43) Pub. Date: Mar. 12, 2026

(54) INHIBITORS OF BROMODOMAIN-CONTAINING PROTEIN 4 AND PHOSPHOINOSITIDE 3-KINASE

(71) Applicant: Board of Regents of the University of Nebraska, Lincoln, NE (US)

(72) Inventors: Ram I. Mahato, Omaha, NE (US); Virender Kumar, Omaha, NE (US); Yuxiang Dong, Omaha, NE (US); Bharti Sethi, Omaha, NE (US)

(21) Appl. No.: 19/105,170

(22) PCT Filed: Sep. 1, 2023
(86) PCT No.: PCT/US23/73282

§ 371 Date: Feb. 20, 2025

Related U.S. Application Data
(60) Provisional application No. 63/403,099, filed on Sep. 1, 2022.

(51) Int. Cl. A61K 31/5377; A61K 31/353; A61K 31/366; A61K 45/06; A61P 35/00; C07D 311/22; C07D 319/18; C07D 407/04; C07D 407/12; C07D 407/14; C07D 413/10; C07D 413/14; C07D 471/04; C07D 487/04

(52) U.S. Cl. CPC A61K 31/5377; A61K 31/353; A61K 31/366; A61K 45/06; A61P 35/00; C07D 311/22; C07D 319/18; C07D 407/04; C07D 407/12; C07D 407/14; C07D 413/10; C07D 413/14; C07D 471/04; C07D 487/04

(57) ABSTRACT

Methods and compositions for treating, inhibiting, and/or preventing diseases or disorders associated with aberrant bromodomain-containing protein 4 (BRD4) and phosphoinositide 3-kinase (PBK) activity are disclosed, the methods comprise contacting a solution, cell, tissue, or subject expressing BRD4 and PBK with a compound having Benzopyran-4-One core.

This application claims priority under 35 U.S.C. § 119(e) to U.S. Provisional Patent Application No. 63/403,099, filed on Sep. 1, 2022. The foregoing application is incorporated by reference herein.

This invention was made with government support under Grant No. R01 NS128336 awarded by the National Institutes of Health (NIH). The government has certain rights in the invention.

### FIELD OF THE INVENTION

The present invention relates to the field of cancer. More specifically, the invention provides compositions and methods for the treatment of diseases or disorders associated with aberrant bromodomain-containing protein 4 (BRD4) and phosphoinositide 3-kinase (PI3K) activity.

### BACKGROUND OF THE INVENTION

Several publications and patent documents are cited throughout the specification in order to describe the state of the art to which this invention pertains. Each of these citations is incorporated herein by reference as though set forth in full. Medulloblastoma (MB) is the most common childhood brain tumor arising from the cerebellum. MB has four major dysregulated signaling pathways that form the primary basis of differentiation: sonic hedgehog (SHH), WNT, MYC (Group 3), and Group 4 (Kumar, et al. (2017) Trends Pharmacol. Sci., 38:1061-1084). These molecular subgroupings influence the treatment decision, and each type has a distinct prognosis. Further, with increased molecular discoveries and data from patient samples, these subgroups of MB have been refined into numerous subtypes. Several mutations are known to promote MB development. For instance, mutations in WNT signaling pathway gene APC (Turcot syndrome) may develop into WNT MB.

Similarly, mutations in SHH pathway genes such as PTCH1 (Gorlin syndrome), SUFU, TP53 (Li-Fraumeni syndrome), or SMO (Curry-Jones syndrome) may initiate SHH MB. Group 3 and 4 MBs are more genetically heterogeneous and not driven by well-defined signaling pathways like WNT and SHH MBs.

Group 4 is the most frequent MB, which represents 35% of all MBs, and exhibits an intermediate prognosis. Group 4 MB is characterized by a single copy gain of the SNCAIP gene in mutual exclusion with MYCN and CDK6 amplification in 5-10% of patients. MYC activity is upregulated in almost all MB subtypes and MYC targeting is speculated as a possible therapeutic strategy (Roussel, et al. (2013) Cold Spring Harb. Perspect. Med., 3: a014308).

Overexpression of the MYCN gene is associated with group 3 MB and significantly reduced survival outcomes (Borgenvik, et al. (2020) Front. Oncol., 10:626751). MYC activation develops because of amplification at the MYC loci, genomic rearrangement of PVT1-MYC and/or some-unknown mechanisms (Olivero, et al. (2020) Mol. Cell 77:761-774).

The MYC oncogene family comprises three members: C-MYC, MYCN, and MYCL. MYC is a “super-transcription factor” that controls the transcription of more than 15% of the human genome and enhances the transcription of various genes involved in ribosome biogenesis, protein translation, cell-cycle progression, and metabolism, among others (Chen, et al. (2018) Signal Transduct Target Ther., 3:5). MYC focal amplification locus at 8q24 is significantly associated with tumor aggressiveness and poor clinical outcome (Endersby, et al. (2021) Sci. Transl. Med., 13:eaba7401; Korshunov, et al. (2012) Acta Neuropathol., 123:515-527). Targeting MYC directly has been difficult due to the lack of a specific binding site in its protein. However, inhibiting its upstream targeting proteins such as Bromo- and Extra-terminal domains (BET), histone deacetylases (HDAC), and histone methyltransferase for H4 lysine 20 (SETD8) provide a significant anti-tumor effect in preclinical models of MYC-amplified MB (Roussel, et al. (2013) Cold Spring Harb. Perspect. Med., 3: a014308; Veo, et al. (2019) JCI Insight 4: e122933).

The BET family has four members including BRD2, BRD3, BRD4, and BRDT that play crucial roles in regulating gene transcription through epigenetic interactions between bromodomains and acetylated histones through the activity of their tandem bromodomains (BD1, BD2). The BET proteins share a typical tertiary structure, consisting of four α-helixes (αZ, αA, αB, and αC) and two loops (ZA and BC). The four α-helixes form a left-handed α-helical bundle which, along with the two loops, creates a binding pocket for recognition of acetylated lysine residues on N-terminal histone tails (Devaiah, et al. (2016) Nat. Struct. Mol. Biol., 23:540-548). Published structures exhibit ligands bound to the acetyl-lysine binding pocket that is further stabilized by a water channel located at the back of the acetyl-lysine binding pocket, which are conserved in all the BRD family proteins. Human BRD2 protein is a nuclear Ser/Thr-Kinase whose activity is increased upon cellular proliferation. It also promotes E2F-dependent cell cycle progression. BRD3 directly interacts with acetyl-lysine residues of the transcription factor GATA1 and regulates the expression of all erythroid and megakaryocyte-specific genes (Lamonica, et al. (2011) Proc Natl Acad Sci., 108:E159-168). BRD4 interacts with CDK9 and cyclinT1, constituting the core positive transcription elongation factor b (P-TEFb) which subsequently releases RNA Pol II from pausing in the promoter-proximal region (Taniguchi, Y. (2016) Int. J. Mol. Sci., 17:1849). Among these BET proteins, BRD4 upregulation is well-documented in various cancers. In recent years, numerous BRD4 inhibitors have entered clinical trials and achieved significant results in tumor treatment. BRD4 inhibition by (+)-JQ1 decreases the growth of c-MYC driven tumors (Wang, et al. (2020) J. Control Release 323:463-474). However, (+)-JQ1 (1) is non-selective towards all BET family members, which produces significant side effects (Zhou, et al. (2020) Front. Pharmacol., 11:1043).

BRD4 inhibition not only cures patients but also overcomes resistance due to the WNT/-catenin pathway, IGF-I, PDGF, HGF, and neurotrophins (Guerreiro, et al. (2008) Clin. Cancer Res., 14:6761-6769). Additionally, mutation of phosphatidylinositol-4,5-bisphosphate 3-kinase catalytic subunit alpha in SHH-MB has been shown to accelerate the growth and metastasis of MB in mice. Activation of different receptor tyrosine kinases (RTK) such as IGF-IR, PDGF receptor (PDGFR), Trk, and fibroblast growth factor receptor (FGFR) converge into the PI3K signaling pathway, which plays a crucial role in controlling cell proliferation, survival, and motility/metastasis. Therefore, targeting any individual receptors may fail to provide therapeutic benefits.

Further, mutations in the catalytic subunit alpha of PI3K have been shown to accelerate the tumor growth in SHH-MB and metastasis of MB in mice (Marcotte, et al. (2016) Cell 164:293-309). PI3K activates AKT and mTOR to enhance mRNA translation and increases MYC protein half-life and MYC transcriptional activity. Therefore, targeting any individual receptor may fail to provide therapeutic benefits, but directly targeting PI3K represents a potentially successful therapeutic strategy and is under investigation in different cancers (Yang, et al. (2019) Mol. Cancer 18:26). Nonetheless, improved methods of treating MB are needed.

### SUMMARY OF THE INVENTION

In accordance with one aspect of the instant invention, compounds which are dual inhibitors of BRD4 and PI3K are provided. In certain embodiments, the compound is of formula (I), or a pharmaceutically acceptable salt thereof. In certain embodiments, the compound is MDP5. Compositions comprising a compound of the instant invention and a carrier, particularly a pharmaceutically acceptable carrier, are provided.

In accordance with another aspect of the instant invention, methods of inhibiting and/or reducing BRD4 activity and PI3K activity are provided. In certain embodiments, the method comprises contacting BRD4 and PI3K with a compound of the instant invention. In certain embodiments, the method comprises contacting a solution, cell, tissue, or subject comprising or expressing BRD4 and PI3K with a compound of the instant invention.

In accordance with another aspect of the instant invention, methods of treating, inhibiting, and/or preventing a disease or disorder characterized by aberrant BRD4 and PI3K activity are provided. In certain embodiments, the method comprises administering a therapeutically effective amount of a compound of the instant invention to a subject in need thereof. In certain embodiments, the disease or disorder is cancer. In certain embodiments, the disease or disorder is a fibrotic disease. In certain embodiments, the disease or disorder is liver cirrhosis.

### BRIEF DESCRIPTION OF THE DRAWINGS

FIG. 1A provides chemical structures and names of the MDP series compounds. FIG. 1B provides a schematic of the synthesis scheme of MDP5. Briefly, MDP5 was synthesized in a two-step sequence that involves the reaction of a salicylic acid with N-acetylmorpholine/POCl₃, followed by the Suzuki coupling reaction of the arylbromide intermediate with an arylboronic acid.

FIGS. 2A-2D show the effect of MDP5 treatment on cell viability and migration potential in MB cells. FIGS. 2A and 2B provide graphs of cell viability assays with DAOY cells (FIG. 2A) and HD-MB03 cells (FIG. 2B) with the indicated compounds. FIG. 2C provides images of the Transwell® migration assay and FIG. 2D provides quantitation of the Transwell® migration assay (top: DAOY cells; bottom: HD-MB03 cells). Cells were fixed in formalin and stained with 0.5% crystal violet for 30 minutes. Images were taken using a microscope at 20× objective. Stained cells were then dissolved in acetic acid solution and optical density (OD) was measured at 590 nm. Scale bar: 100 μm. **P<0.01 and ***P<0.001 vs Control.

FIGS. 3A-3F show the effect of MDP5 treatment on cell cycle analysis, induction of apoptosis, and colony formation assay in MB cells. DAOY and HD-MB03 cells were treated with SF2523, MDP1, and MDP5 at 5 μM for 48 hours. Histograms show the quantitative analysis of cell cycle assay for DAOY (FIG. 3A) and HD-MB03 (FIG. 3B) cells (n=5; mean±S.D., p<0.05). Cells treated with SF2523 and MDP5 show cycle arrest in G1 phase as compared to control and MDP1. FIG. 3C provides images of cells treated with the indicated compounds. MDP5 treatment showed a lower number of colonies in both cell lines. FIGS. 3D and 3E provide graphs of the apoptotic assay showing higher numbers of apoptotic cells in MDP5 treated groups (n=5; mean±S.D.; p<0.05). FIG. 3F provides a graph of the relative expression of BCL2 and MYCN target genes after treatment, as determined by RT-PCR.

FIGS. 4A-4G show that BRD4 and PI3K dual inhibitor MDP5 treatment decreases target gene expression at protein level. FIGS. 4A and 4B provide images of western blot analyses of target proteins after HDMB03 and DAOY cells were incubated with 5.0 μM of the indicated compounds for 48 hours. FIG. 4C (HD-MB03) and 4D (DAOY) provide western blot images which show the effects of MDP5 treatment on target proteins including GLI1 and GLI2. FIG. 4E provides representative confocal images of DAOY and HD-MB03 spheroids after treatment with 5.0 μM MDP5. MDP5 decreased the tumor spheroid formation in both MB cells. Spheroids were stained for live (calcein AM) and dead (ethidium homodimer-1) cells after 7 days of treatment. Scale bars=200 μm. FIG. 4F provides images of FACS analysis which shows the effect of MDP5 treatment on CD15+ population in MB cells ONS-76. SHH group (ONS-76 cells) were treated with SF2523, MDP1, and MDP5 for 48 hours at 5.0 μM. Next, cells were incubated with APC-CD15 for 30 minutes on ice in the dark. Before FACS analysis, samples were washed thrice with a staining buffer. FIG. 4G provides a graph showing the quantitation of CD15+ cells (n=3; p<0.05, control vs. treatment groups).

FIGS. 5A-5G show the results of the in vivo efficacy study in the MB xenograft model. FIG. 5A provides a graph of the animal's body weight during the treatment period. FIG. 5B provides a graph of the tumor burden curve of DAOY cell-generated tumors. FIG. 5C provides representative images of harvested DAOY cell-generated tumors. FIG. 5D provides a graph of tumor weights (mean±S.D., n=5, p<0.05). FIG. 5E provides a graph of the tumor burden curve of HD-MB03 cell generated tumors. FIG. 5F provides representative images of harvested HD-MB03 cell-generated tumors. FIG. 5G provides a graph of HD-MB03 generated tumor weights (mean±S.D., n=5, p<0.05).

FIGS. 6A-6C show the in vivo efficacy study in DAOY cell generated orthotopic MB-bearing NSG mice. FIG. 6A provides bioluminescence images and FIG. 6B provides a quantitative analysis of IVIS signal intensity (photons/s/cm²/sr) over time during the treatment with MDP5. FIG. 6C provides a Kaplan-Meier survival curve.

### DETAILED DESCRIPTION OF THE INVENTION

MYC oncogene is upregulated in a variety of human cancers. Since direct MYC inhibition has been proven a difficult therapeutic target, the inhibition of MYC upstream genes such as the IGF/PI3K signaling pathway has been a major focus of anticancer drug development. However, inhibition of PI3K to enhance MYC degradation provides only limited therapeutic benefits and often leads to the activation of compensatory pathways and chemoresistance. These resistance mechanisms can be overcome by simultaneously inhibiting BRD4, a transcription enhancer for MYC and GLI genes. However, inhibiting these two pathways using two different drugs can lead to an excess of toxicity and differential spatiotemporal effect(s).

The BRD4 and PI3K/AKT dual inhibitor SF2523 reduced MB growth in mice (Kumar, et al. (2021) Biomaterials 278:121138). Since SF2523 has a modest potency, delivery of a sufficient dose to the brain may be challenging and higher doses could lead to systemic toxicity. Therefore, new dual BRD4 and PI3K/AKT inhibitors, including 8-(2,3-dihydrobenzo[b] [1,4] dioxin-6-yl)-2-morpholino-4H-chromen-4one (MDP5), are provided herein. X-ray crystal structures of BRD2-BD1/MDP5, BRD2-BD2/MDP5, BRD2-BD2/SF2523, and BRD4-BD2/SF2523 highlight interactions between MDP5 and SF2523 with bromodomains from both BRD2 and BRD4 as well as supporting MDP5 selection as the lead over the other MDP derivatives. MDP5 showed higher potency in DOAY cells (IC₅₀ 5.5 μM) compared to its parent compound SF2523 (IC₅₀ 12.6 μM), and its IC₅₀ values in HD-MB03 MB (MYC amplified) cells were similar to that observed with SF2523. MDP5 decreased cellular levels of downstream proteins like p-AKT, MYCN, and cyclin D1 while increasing the degradation of MYCN protein. Treatment of MB cells with MDP5 significantly decreased the colony formation capacity, increased apoptosis, and disturbed cell cycle progression. Further, MDP5 (20 mg/kg) was well tolerated (no bodyweight change) in NSG mice bearing xenograft MB generated using DAOY cells. Treatment with MDP5 reduced tumor growth compared to the control group measured by tumor volume. MDP5 treatment also prolonged the survival in an orthotopic MB model.

Herein, novel bromodomain-containing protein 4 (BRD4) and phosphoinositide 3-kinase (PI3K) inhibitors are provided. Methods of inhibiting BRD4 and PI3K and methods of inhibiting, treating, and/or preventing a disorder in a subject associated with aberrant and/or dysregulated BRD4 and PI3K activity are also provided. PI3K activates the downstream protein mechanistic target of rapamycin (mTOR), responsible for tumor cell proliferation, resistance, and survival. BRD4 is mostly acknowledged in cancer for its role in super-enhancers organization and regulation of oncogene expression. Together, the dysregulation of the PI3K signaling pathway and the hyperactivity of transcriptional and epigenetic regulator BRD4 promote cancer development. Simultaneous targeting of BRD4 and PI3K pathways is effective for the treatment of human cancers and fibrotic diseases.

In accordance with the instant invention, compounds of Formula (I) are provided, including pharmaceutically acceptable salts and stereoisomers of the compound. Compounds of Formula (I) are inhibitors of BRD4 and PI3K.

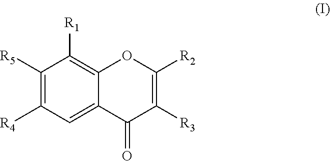

wherein:
R₁ is selected from the group consisting of halo alkyl benzodioxine, halo, alkyl benzodioxine, H, trialkyl isoxazole, dialkylisoxazole, haloalkoxy alkylpyridine alkylbenzamide, benzodioxane alkylamine, aryl, benzodioxane, amino benzodioxane, and imidazo pyridazine alkyne, wherein each group may be optionally substituted;
R₂ is selected from the group consisting of H, alkyl morpholine, morpholinyl, trihalophenoxy, aryl, and trihaloanisole, wherein each group may be optionally substituted;
R₃ is selected from the group consisting of alkyl morpholine, morpholinyl, H, and halo, wherein each group may be optionally substituted;
R₄ is selected from the group consisting of H, alkyl benzodioxine, alkoxy alkylbenzene, halo, trialkylisoxazole, dialkylisoxazole, imidazopyridazine, aryl, benzyloxy, and alkyl, wherein each group may be optionally substituted; and
R₅ is H, benzodioxane or an aryl, wherein each group may be optionally substituted.

In certain embodiments, R₁ is selected from the group consisting of optionally substituted benzodioxane, halo, dialkylisoxazole, 3,5-dimethylisoxazol-4-yl, N-benzyl-4-fluoro-5-methoxybenzamide, H, and amino-1,4-benzodioxane. In certain embodiments, R₁ is N-benzyl-4-fluoro-5-methoxybenzamide. In certain embodiments, R₁ is dialkylisoxazole, particularly wherein the alkyl is a lower alkyl. In certain embodiments, R₁ is 3,5-dialkylisoxazole. In certain embodiments, R₁ is 3,5-dimethylisoxazol-4-yl. In certain embodiments, R₁ is halo, particularly Br. In certain embodiments, R₁ is amino-1,4-benzodioxane. In certain embodiments, R₁ is 1,4-benzodioxane. In certain embodiments, R₁ is 2,3-dihydrobenzo[b][1,4] dioxin-6-yl. In certain embodiments, R₁ is 7-halo-2,3-dihydrobenzo[b][1,4] dioxin-6-yl. In certain embodiments, R₁ is 7-bromo-2,3-dihydrobenzo[b][1,4] dioxin-6-yl. In certain embodiments, R₁ is

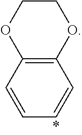

In certain embodiments, R₂ is selected from the group consisting of H, halo, morpholinyl, and trihalophenoxy. In certain embodiments, R₂ is morpholinyl. In certain embodiments, R₂ is

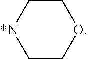

In certain embodiments, R₂ is trihalophenoxy. In certain embodiments, R₂ is 2,4,6-trihalophenoxy. In certain embodiments, R₂ is tribromophenoxy. In certain embodiments, R₂ is 2,4,6-tribromophenoxy. In certain embodiments, R₂ is

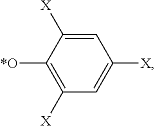

wherein X is halo, particularly Br.

In certain embodiments, R₃ is selected from the group consisting of H, halo, and morpholinyl. In certain embodiments, R₃ is H. In certain embodiments, R₃ is halo, particularly Cl. In certain embodiments, R₃ is morpholinyl. In certain embodiments, R₃ is

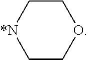

In certain embodiments, R₄ is selected from the group consisting of H, halo, phenyl alkoxy, an optionally substituted benzodioxane, dialkylisoxazole, 3,5-dimethylisoxazol-4-yl, alkyl (e.g., lower alkyl) and imidazopyridazine (e.g., imidazo [1,2-b] pyridazin-3-yl). In certain embodiments, R₄ is H. In certain embodiments, R₄ is benzyloxy. In certain embodiments, R₄ is

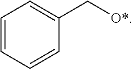

In certain embodiments, R₄ is dialkylisoxazole, particularly wherein the alkyl is a lower alkyl. In certain embodiments, R₄ is 3,5-dialkylisoxazole. In certain embodiments, R₄ is 3,5-dimethylisoxazol-4-yl. In certain embodiments, R₄ is halo, particularly Cl. In certain embodiments, R₄ is alkyl, particularly lower alkyl, particularly methyl. In certain embodiments, R₄ is imidazo [1,2-b] pyridazin-3-yl. In certain embodiments, R₅ is 1,4-benzodioxane. In certain embodiments, R₅ is 2,3-dihydrobenzo [b][1,4]dioxin-6-yl. In certain embodiments, R₅ is 7-halo-2,3-dihydrobenzo [b][1,4] dioxin-6-yl. In certain embodiments, R₅ is 7-bromo-2,3-dihydrobenzo [b][1,4] dioxin-6-yl. In certain embodiments, R₅ is

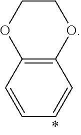

In certain embodiments, R₅ is H or an optionally substituted benzodioxane. In certain embodiments, R₅ is H. In certain embodiments, R₅ is 1,4-benzodioxane. In certain embodiments, R₅ is 2,3-dihydrobenzo [b][1,4] dioxin-6-yl. In certain embodiments, R₅ is 7-halo-2,3-dihydrobenzo [b][1,4] dioxin-6-yl. In certain embodiments, R₅ is 7-bromo-2,3-dihydrobenzo [b][1,4] dioxin-6-yl. In certain embodiments, R₅ is

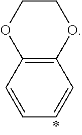

In certain embodiments, R₂, R₄, and R₅ are H or halo. In certain embodiments, R₂, R₄, and R₅ are H. In certain embodiments, R₂, R₄, and R₅ are H; R₁ is selected from the group consisting of halo alkyl benzodioxine, halo, alkyl benzodioxine, H, trialkyl isoxazole, haloalkoxy alkylpyridine alkylbenzamide, benzodioxane alkylamine, an optionally substituted aryl, and imidazo pyridazine alkyne; and R₃ is selected from the group consisting of alkyl morpholine, morpholinyl, H, and halo. In certain embodiments, R₂, R₄, and R₅ are H; R₁ is selected from the group consisting of optionally substituted benzodioxane, halo, dialkylisoxazole, 3,5-dimethylisoxazol-4-yl, N-benzyl-4-fluoro-5-methoxybenzamide, and amino-1,4-benzodioxane; and R₃ is selected from the group consisting of H, halo, and morpholinyl. In certain embodiments, R₂, R₄, and R₅ are H; R₃ is morpholinyl; and R₁ is an optionally substituted 1,4-benzodioxane. In certain embodiments, R₃ is

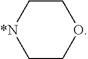

In certain embodiments, R₁ is 1,4-benzodioxane. In certain embodiments, R₁ is 2,3-dihydrobenzo [b][1,4] dioxin-6-yl. In certain embodiments, R₁ is 7-halo-2,3-dihydrobenzo [b][1,4] dioxin-6-yl. In certain embodiments, R₁ is 7-bromo-2,3-dihydrobenzo [b][1,4] dioxin-6-yl. In certain embodiments, R₁ is

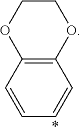

In certain embodiments, compounds of the present invention include, but are not limited to:

### 8-(7-bromo-2,3-dihydrobenzo [b][1,4] dioxin-6-yl)-3-morpholino-4H-chromen-4-one (MDP2)

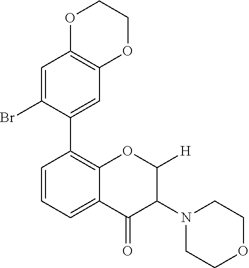 [SMILES: O=C1c2cccc(-c3cc4c(cc3Br)OCCO4)c2OCC1N1CCOCC1]

### 8-(7-bromo-2,3-dihydrobenzo [b][1,4] dioxin-6-yl)-2-morpholino-4H-chromen-4-one

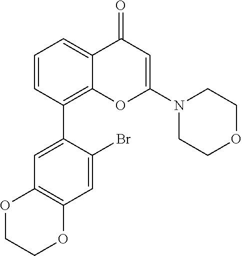 [SMILES: O=c1cc(N2CCOCC2)oc2c(-c3cc4c(cc3Br)OCCO4)cccc12]

### 8-bromo-2-morpholino-4H-chromen-4-one (MDP4)

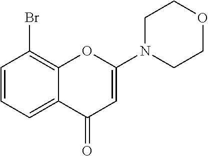 [SMILES: O=c1cc(N2CCOCC2)oc2c(Br)cccc12]

### 6-(benzyloxy)-8-(2,3-dihydrobenzo [b][1,4] dioxin-6-yl)-2-morpholino-4H-chromen-4-one

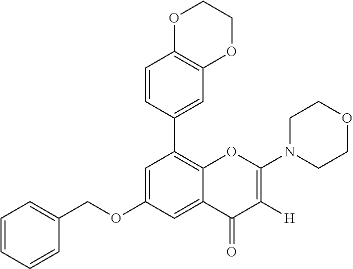 [SMILES: O=c1cc(N2CCOCC2)oc2c(-c3ccc4c(c3)OCCO4)cc(OCc3ccccc3)cc12]

### 6,8-bis(2,3-dihydrobenzo [b][1,4] dioxin-6-yl)-2-morpholino-4H-chromen-4-one

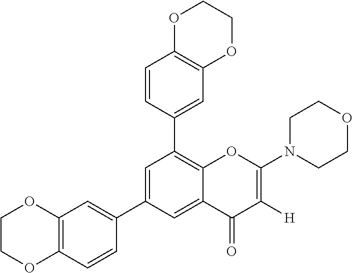 [SMILES: O=c1cc(N2CCOCC2)oc2c(-c3ccc4c(c3)OCCO4)cc(-c3ccc4c(c3)OCCO4)cc12]

### 8-(2,3-dihydrobenzo [b][1,4] dioxin-6-yl)-2-morpholino-4H-chromen-4-one (MDP5)

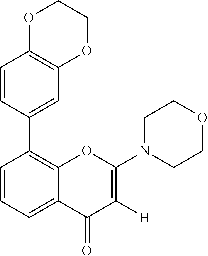 [SMILES: O=c1cc(N2CCOCC2)oc2c(-c3ccc4c(c3)OCCO4)cccc12]

### 7-(2,3-dihydrobenzo [b][1,4] dioxin-6-yl)-2-morpholino-4H-chromen-4-one

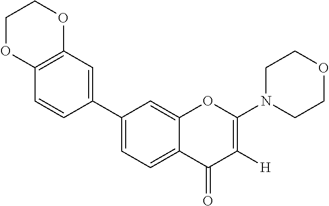 [SMILES: O=c1cc(N2CCOCC2)oc2cc(-c3ccc4c(c3)OCCO4)ccc12]

### 8-bromo-6-chloro-2-(2,4,6-tribromophenoxy)-4H-chromen-4-one

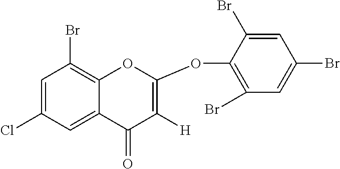 [SMILES: O=c1cc(Oc2c(Br)cc(Br)cc2Br)oc2c(Br)cc(Cl)cc12]

### 6,8-bis(3,5-dimethylisoxazol-4-yl)-2-morpholino-4H-chromen-4-one

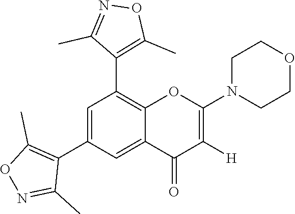 [SMILES: Cc1noc(C)c1-c1cc(-c2c(C)noc2C)c2oc(N3CCOCC3)cc(=O)c2c1]

### 6-chloro-8-(3,5-dimethylisoxazol-4-yl)-2-morpholino-4H-chromen-4-one

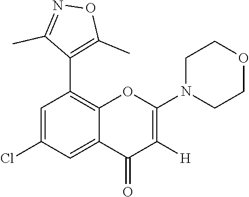 [SMILES: Cc1noc(C)c1-c1cc(Cl)cc2c(=O)cc(N3CCOCC3)oc12]

### N-benzyl-3-(6-chloro-2-morpholino-4-oxo-4H-chromen-8-yl)-4-fluoro-5-methoxybenzamide

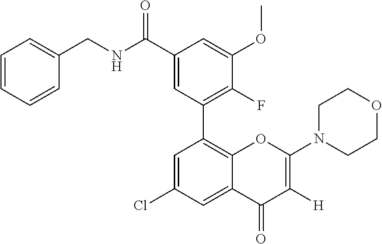 [SMILES: COc1cc(C(=O)NCc2ccccc2)cc(-c2cc(Cl)cc3c(=O)cc(N4CCOCC4)oc23)c1F]

### 6-chloro-8-((2,3-dihydrobenzo [b][1,4] dioxin-6-yl) amino)-2-morpholino-4H-chromen-4-one

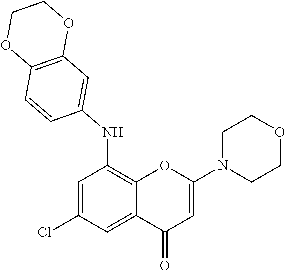 [SMILES: O=c1cc(N2CCOCC2)oc2c(Nc3ccc4c(c3)OCCO4)cc(Cl)cc12]

### 8-((2,3-dihydrobenzo [b][1,4] dioxin-6-yl) amino)-2-morpholino-4H-chromen-4-one

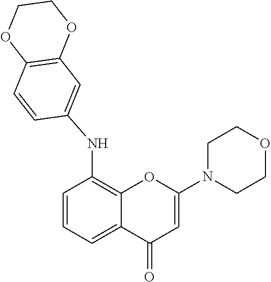 [SMILES: O=c1cc(N2CCOCC2)oc2c(Nc3ccc4c(c3)OCCO4)cccc12]

### 8-(2,3-dihydrobenzo [b][1,4] dioxin-6-yl)-6-methyl-2-morpholino-4H-chromen-4-one

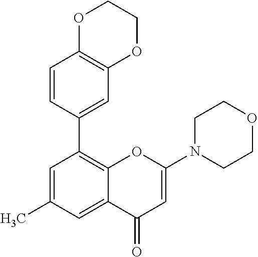 [SMILES: Cc1cc(-c2ccc3c(c2)OCCO3)c2oc(N3CCOCC3)cc(=O)c2c1]

### 3-chloro-8-(2,3-dihydrobenzo [b][1,4] dioxin-6-yl)-2-morpholino-4H-chromen-4-one

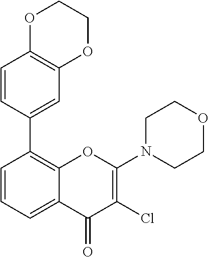 [SMILES: O=c1c(Cl)c(N2CCOCC2)oc2c(-c3ccc4c(c3)OCCO4)cccc12]

### 6-(imidazo [1,2-b] pyridazin-3-yl)-2-morpholino-4H-chromen-4-one

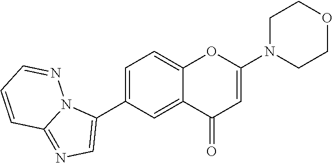 [SMILES: O=c1cc(N2CCOCC2)oc2ccc(-c3cnc4cccnn34)cc12]

Compounds of the present invention may additionally have any of the following structures:

### (E)-1-(3-(7-bromo-2,3-dihydrobenzo [b][1,4] dioxin-6-yl)-2-hydroxyphenyl)-3-morpholinoprop-2-en-1-one (MDP1)

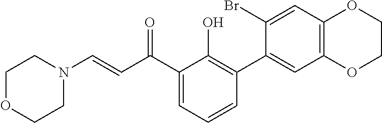 [SMILES: O=C(/C=C/N1CCOCC1)c1cccc(-c2cc3c(cc2Br)OCCO3)c1O]

### 1-bromo-3-morpholino-6,7-dihydro-4H-pyrazino [2,1-a]isoquinolin-4-one (MDP3)

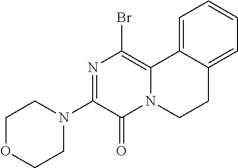 [SMILES: O=c1c(N2CCOCC2)nc(Br)c2n1CCc1ccccc1-2]

### 4,8-bis(2,3-dihydrobenzo [b][1,4] dioxin-6-yl)-3-methyl-2H-chromen-2-one

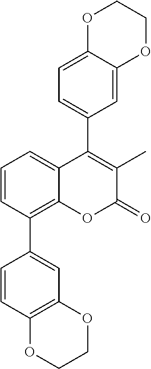 [SMILES: Cc1c(-c2ccc3c(c2)OCCO3)c2cccc(-c3ccc4c(c3)OCCO4)c2oc1=O]

The compounds of the instant invention encompass pharmaceutically acceptable salts and stereoisomers of the compound.

Compositions comprising the compounds of the instant invention are also encompassed. In certain embodiments, the compositions comprise a compound and a carrier, particularly a pharmaceutically acceptable carrier. In certain embodiments, the compositions further comprise an additional therapeutic agent. In certain embodiments, the additional therapeutic agent is an anti-cancer drug (e.g., chemotherapeutic agent), anti-inflammatory drug, or immune-modulatory drug.

In accordance with another aspect of the instant invention, methods of inhibiting BRD4 and/or PI3K are provided. The methods can be performed in vivo or in vitro. In certain embodiments, the method comprises contacting BRD4 and/or PI3K with a compound or composition of the instant invention. In certain embodiments, the method comprises contacting a solution, cell, tissue, and/or subject comprising and/or expressing BRD4 and/or PI3K with a compound or composition of the instant invention. In certain embodiments, the method inhibits and/or reduces BRD4 and/or PI3K activity. In certain embodiments, the method inhibits BRD4-mediated transcriptional activity. In certain embodiments, the method inhibits BRD4 from binding DNA. In certain embodiments, the method inhibits PI3K kinase activity.

In accordance with another aspect of the instant invention, methods of treating, inhibiting, and/or preventing a disease or disorder in a subject in need thereof are provided. The methods comprise administering a compound of the instant invention or a composition comprising a compound of the instant invention to a subject. In certain embodiments, the disease or disorder is associated with aberrant and/or dysregulated BRD4 and/or PI3K activity (e.g., increased BRD4 and/or PI3K activity compared to healthy subjects or inappropriate BRD4 and/or PI3K activity compared to healthy subjects). In certain embodiments, the disease or disorder is a BRD4-driven and PI3K-driven disease or disorder. In certain embodiments, the disease or disorder is fibrotic disease. Examples of fibrotic disease include, without limitation: liver fibrosis (e.g., cirrhosis), lung fibrosis, kidney fibrosis, and heart fibrosis. In certain embodiments, the disease or disorder is cancer. Examples of cancer include, without limitation: prostate cancer, bladder cancer, renal cancer, gastric cancer, liver cancer, pancreatic cancer, colorectal cancer, cancers of the central nervous system (e.g., gliomas, meningiomas, pituitary adenomas, medulloblastoma, and neuroblastoma), breast cancer, melanoma, hematological cancers (e.g., acute myeloid leukemia and other leukemias), lymphomas, multiple myeloma, colon cancer, thyroid cancer, lung cancer, ovarian cancer, stomach cancer, cervical cancer, testicular cancer, kidney cancer, carcinoid tumors, and bone cancer. In certain embodiments, the cancer is medulloblastoma. In certain embodiments, the cancer is acute myeloid leukemia (AML).

In certain embodiments, the methods of the instant invention further comprise administering an additional therapeutic agent to the subject. In certain embodiments, the additional therapeutic agent is an anti-cancer drug (e.g., chemotherapeutic agent), anti-inflammatory drug, or immune-modulatory drug. The additional therapeutic agent can be administered before, after and/or at the same time as the compound or composition of the instant invention. In certain embodiments, the additional therapeutic agent is administered in the same composition as the compound of the instant invention. In certain embodiments, the additional therapeutic agent is administered in a different composition (e.g., comprising a carrier, particularly a pharmaceutically acceptable carrier) as the compound of the instant invention.

The compounds and compositions of the present invention may be conveniently formulated for administration (e.g., to a subject) with any pharmaceutically acceptable carrier(s). The compounds and compositions of the present invention can be administered by any suitable route, for example, by injection (e.g., for local (e.g., direct, including to or within a tumor) or systemic administration), oral, pulmonary, topical, nasal or other modes of administration. The composition may be administered by any suitable means including, without limitation: parenterally, subcutaneously, orally, topically, intrapulmonarily, rectally, vaginally, intrarectally, intravenously, intraperitoneally, intraarterially, intrathecally, inhalation, intranasally, transdermally, intracerebrally, epidurally, intramuscularly, intradermally, or intracarotidly. In a particular embodiment, the compound or composition is administered to the blood (e.g., intravenously). In a particular embodiment, the compound or composition is administered locally to the desired site (e.g., site of treatment, site of tumor, etc.). For example, the compound or composition may be administered by intratumoral injection or by injection near the tumor site. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween® 80, polysorbate 80), antioxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylene-vinyl acetate copolymers, polylactic acid, polyglycolic acid, etc., or into liposomes. Such compositions may influence the physical state, stability, rate of in vivo release, and rate of in vivo clearance of components of a pharmaceutical composition of the present invention (e.g., Remington: The Science and Practice of Pharmacy). The pharmaceutical composition of the present invention can be prepared, for example, in liquid form, or can be frozen (e.g., by freeze drying (optionally with a cryoprotectant); e.g., with the addition of at least one cell-freezing component (e.g., DSMO, serum, cell culture medium, etc.) and placement in liquid nitrogen).

As used herein, “pharmaceutically acceptable carrier” includes all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in pharmaceutical preparation is contemplated.

The dose and dosage regimen of the compound or composition of the invention that is suitable for administration to a particular patient may be determined by a physician considering the patient's age, sex, weight, general medical condition, and the specific condition and severity thereof for which the inhibitor is being administered. The physician may also consider the route of administration, the pharmaceutical carrier, and the compound's biological activity.

Selection of a suitable pharmaceutical preparation depends upon the method of administration chosen. For example, the compounds of the invention may be administered by direct injection into any cancerous tissue or into the area surrounding the cancer. In this instance, a pharmaceutical preparation comprises the compounds dispersed in a medium that is compatible with the cancerous tissue.

Pharmaceutical compositions of the present invention can be prepared according to conventional pharmaceutical compounding techniques. The carrier may take a wide variety of forms depending on the form of preparation desired for administration, e.g., intravenous, oral, topical, or parenteral. For injection or parenterals, the carrier will usually comprise sterile water and salts (e.g., saline), though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain an adequate quantity of the active ingredient calculated to produce the desired effect in association with the selected pharmaceutical carrier. Procedures for determining the appropriate dosage unit are well known to those skilled in the art. Dosage units may be proportionately increased or decreased based on the weight/surface area of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects because of the treatment. Appropriate dosage unit may also be determined by assessing the efficacy of the treatment in combination with other standard chemotherapies. The dosage units of the molecules may be determined individually or in combination with other chemotherapeutic drug or other form of therapy according to greater shrinkage and/or reduced growth rate of tumors.

The pharmaceutical preparation comprising the compounds of the instant invention may be administered at appropriate intervals until the pathological symptoms are cured, reduced or alleviated, after which the dosage may be reduced to a maintenance level, if needed. The appropriate interval in a particular case would normally depend on the condition of the patient.

### Definitions

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in, for example, Remington: The Science and Practice of Pharmacy; Liberman, et al., Eds., Pharmaceutical Dosage Forms; and Rowe, et al., Eds., Handbook of Pharmaceutical Excipients.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., cancer) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof.

As used herein, the term “subject” refers to an animal, particularly a mammal, particularly a human.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; and tubulin interactive agents (e.g., vincristine, vinblastine, and paclitaxel (Taxol®)).

As used herein, the term “alkyl” refers to straight or branched chain, saturated hydrocarbons containing 1 to about 30 carbons in the normal/main chain. In certain embodiments, the alkyl group contains from 1 to 6 carbon atoms. The term “lower alkyl” refers to an alkyl which contains 1 to 3 carbons in the hydrocarbon chain.

Examples of alkyl moieties include, without limitation: methyl, ethyl, n-propyl, isopropyl, n-butyl, tert-butyl, isobutyl, sec-butyl, n-pentyl, 3-pentyl, n-hexyl, 1,2,2-trimethylpropyl, etc.

The terms “halo” or “halogen” refers to fluoro (F), chloro (Cl), bromo (Br) and iodo (I). In certain embodiments, “halo” refers to F, Cl, or Br. In certain embodiments, halo groups are Br or Cl.

The term “aryl”, as used herein, refers to an aromatic hydrocarbon group which is monocyclic or polycyclic (e.g., having 2 or more fused rings wherein at least one ring is aromatic). In certain embodiments, the aryl group comprises about 6 to about 10 carbon atoms. In certain embodiments, the aryl group is a heteroaryl. The term “heteroaryl,” as used herein, refers to an aryl group having at least one heteroatom ring member independently selected from sulfur, oxygen and nitrogen. In certain embodiments, the heteroaryl ring has 1, 2, 3 or 4 heteroatoms.

The term “substituted,” as used herein, means that an atom or group of atoms formally replaces one or more hydrogens as a “substituent” attached to another group. A group may contain any number of substituents. The substituents are independently selected. Examples of substituents include, without limitation, alkyl (e.g., lower alkyl), alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, ether, ester, thioester, nitrile, nitro, amide, carbonyl, carboxylate and thiol.

The following example is provided to illustrate certain embodiments of the invention. It is not intended to limit the invention in any way.

### Example

### Materials and Methods

### Reagents and Cell Culture

SF2523 (HY-101146) and LY294002 (HY-10108) were purchased from MedChem Express LLC. For in vitro studies, all the drugs were dissolved in dimethyl sulfoxide (DMSO) to make the desired stock concentrations. Cell culture media EMEM, DMEM, and RPMI were purchased from the ATCC, Hyclone Laboratories, and Gibco, respectively. FBS was purchased from BioTechne, and an antibiotic solution Anti-biotic-Antimycotic (Anti-Anti) was obtained from Sigma-Aldrich. DAOY, HD-MB03, and ONS-76 MB (or MB) cells were cultured in EMEM, DMEM, and RPMI, respectively, with 10% FBS and 1% Anti-Anti. All cells were maintained in an incubator at 37° C. with relative humidity between 90 and 95% in the presence of 5% CO₂. Primary antibodies were obtained from Abcam, Biomatik, Cell signaling technology (CST), and Santacruz Biotech. Horseradish peroxidase (HRP) conjugated and fluorescent labeled secondary antibodies were purchased from the Invitrogen (A16096) and Li—COR Biosciences (926-68070), respectively. All other reagents were obtained from Fisher Scientific.

### Synthesis of 8-(2,3-dihydrobenzo [b][1,4] dioxin-6-yl)-2-morpholino-4H-chromen-4-one (MDP5)

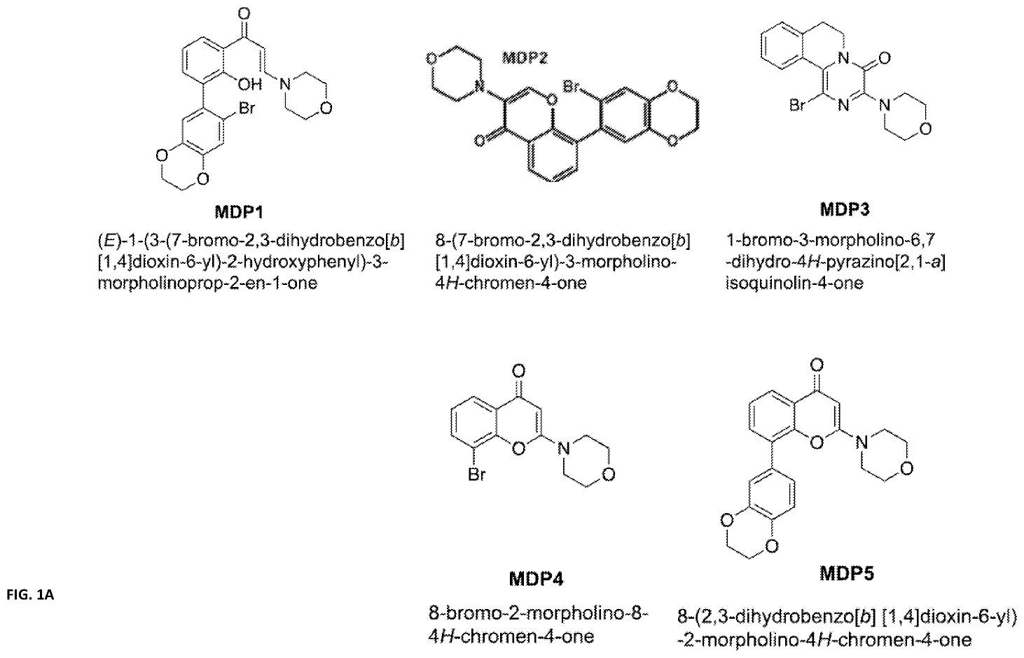

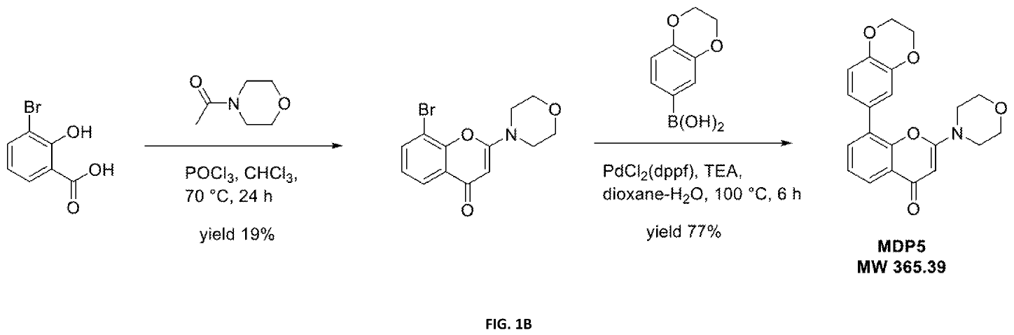

The synthesis of MDP5 was performed in the following two steps. A schematic of the synthesis is provided in FIG. 1B.

Step 1: To a solution of 3-bromo-2-hydroxybenzoic acid (3.48 g, 16 mmol) and N-acetylmorpholine (4.12 g, 32 mmol) in CHCl₃ (10 mL), POCl₃ (6 mL, 64 mmol) was added. The mixture was stirred at RT for 1 hour and then heated at 70° C. for 24 hours. After the reaction mixture was cooled to room temperature (RT), it was quenched with water (50 mL), CHCl₃ (100 mL), and NaOAc (25 g, 305 mmol). The mixture was heated at 70° C. for 1 hour and then cooled to RT. After the separation of the organic layer, the aqueous layer was extracted with CHCl₃ (2×20 mL). The combined organic layer was washed with brine (100 mL) and then with a mixture of brine (50 mL) and 10% aq. NaOH (20 mL), dried over MgSO₄, filtered, and concentrated. The residue was purified by crystallization from MTBE to give the chromenone intermediate (945 mg, 19%) as a yellowish solid. ¹H NMR (500 MHz, CDCl3) 8.10 (d, J=8.0 Hz, 1H), 7.77 (d, J=8.0 Hz, 1H), 7.23 (t, J=8.0 Hz, 1H), 5.50 (s, 1H), 3.86 (t, J=5.0 Hz, 4H), 3.59 (t, J=5.0 Hz, 4H).

Step 2: To a mixture of the above chromenone (ArBr, 310 mg, 1 mmol) and 1,4-benzodioxane-6-boronic acid (ArB(OH)2, 360 mg, 2 mmol), and triethanolamine (TEA, 303 mg, 3 mmol) in dioxane (10 mL) and water (4 mL), PdCl₂(DPPF) (40 mg, 0.055 mmol) was added under nitrogen (N₂). The mixture was heated at 100° C. for 6 hours and then cooled to RT. The mixture was diluted with water (50 mL) and then extracted with EtOAc (3×20 mL). The combined organic layer was washed with brine (50 mL), dried over MgSO₄, filtered, and concentrated. The crude product was purified by chromatography (silica gel, EtOAc/EtOH=3:1) followed by crystallization from ether to afford the desired product MDP5 (281 mg, 77%) as a yellowish solid. Mp 182-184° C. ¹H NMR (500 MHz, CDCl₃) 8.13 (d, J=8.0 Hz, 1H), 7.53 (d, J=8.0 Hz, 1H), 7.38 (t, J=7.5 Hz, 1H), 7.05 (d, J=1.5 Hz, 1H), 7.00 (dd, J=7.5, 1.5 Hz, 1H), 6.94 (d, J=8.0 Hz, 1H), 5.51 (s, 1H), 4.29-4.33 (m, 4H), 3.76 (t, J=5.0 Hz, 4H), 3.40 (t, J=5.0 Hz, 4H); ¹³C NMR (125.7 MHz, CDCl₃) 177.28, 162.52, 150.53, 143.47, 143.45, 133.25, 129.71, 129.46, 124.68, 124.67, 123.35, 122.47, 118.37, 117.08, 86.96, 65.94, 64.49, 64.36, 44.78.

### Molecular Docking

The docking study of LY294002, quercetin, SF2523, SF2535, and MDP1-MDP5 was carried out using the Glide docking approach (Protein Preparation Wizard, Maestro, MacroModel, and Glide; Schrödinger, LLC: Portland, OR). BRD4 first bromodomain (BD1, PDB ID: 2U28), BRD4 second bromodomain (BD2, PDB ID: 5U2C), and the PI3Kα subtype protein human p110α/p85α (PDB ID: 2RD0) were retrieved through Protein Data Bank (PDB) (rcsb.org) and were prepared and minimized using the MOE protein preparation module (Kumar, et al. (2021) Biomaterials 278:121138; Andrews, et al. (2017) Proc Natl Acad Sci., 114:E1072-E1080). PI3K protein 2RD0 was prepared as outlined (Sabbah, et al. (2010) J. Chem. Inf. Model 50:1887-1898). The minimized proteins were imported to Maestro (Maestro and Glide dock reference) using the OPLS_2005 force field with backbone atoms restrained to reduce steric repulsion and, in the meantime, to minimize the impact on the backbone atoms. Structures of LY294002, quercetin, SF2523, SF2535, ABBV_744, IBET_762, and MDP1-MDP5 were built and minimized with MMFF force field using MOE program. The minimized structures were used to prepare the Grid files that identify and describe the binding pocket using the Glide Grid Generation protocol with the bound ligand as the centroid.

Ligand structures of LY294002, quercetin, SF2523, ABBV_744, IBET_762, and MDP1-MDP5 were docked to three model proteins as defined by the grid file. During the docking process, the scaling factor for receptor van der Waals for the nonpolar atoms was set to 0.8 to allow for a certain degree of receptor flexibility, and the extra-precision method was used. All other parameters were used as defaults. The binding affinity of the protein/ligand complexes was expressed as docking scores, where ligands with the more negative docking score have strong binding. The protein/ligand interaction figures were generated with the PyMOL program (PyMOL, Delano Scientific LLC, San Carlos, CA), and H-atoms were hidden for clarity purposes.

### BRD2-BD2/BRD4-BD2 Molecular Cloning, Expression, and Purification

The genes encoding human BRD2-BD1, BRD2-BD2, and BRD4-BD2 were codon-optimized for expression in E. coli. The codon-optimized genes were amplified by PCR, and the resulting PCR products were placed downstream of the ribosome binding site of a derivatized pET32 plasmid allowing for the production of a protein possessing a cleavable polyhistidine tag at the N-terminus. The sequence-confirmed plasmids were used to transform T7 express E coli. Large-scale cell cultures were grown in LB media containing 264 mM carbenicillin and incubated with shaking at 37° C. until the OD₆₀₀ nm reached 0.6. Then cultures were cooled to 16° C., and 1 mM isopropyl β-D-1-thiogalactopyranoside was added to induce the protein expression. After 20 hours of incubation, cells were harvested. The resulting cell pellet was resuspended in a lysis buffer containing 50 mM Tris pH 7.5, 200 mM NaCl, 0.5 mM imidazole, and 0.3 mM tris(2-carboxyethyl) phosphine (TECP). After the addition of DNase I and lysozyme, the cell suspension was incubated on ice for 30 minutes and lysed by sonication. The lysate was clarified by centrifugation at 12,000 rpm for 45 minutes. The resulting supernatant was applied to a 5 mL metal affinity cobalt column pre-equilibrated with lysis buffer. Then, 25 column volumes of lysis buffer were passed through the column to eliminate unbound E coli proteins. Recombinant protein was eluted isocratically with the elution buffer containing 50 mM Tris pH 7.5, 200 mM NaCl, 150 mM imidazole, and 0.3 mM TECP. The rhinovirus 3C protease was added to the eluted recombinant protein to cleave the N-terminal Histidine tag and the sample dialyzed against a buffer containing 50 mM Tris pH 7.5, 100 mM NaCl and 0.3 mM TECP. After 12 hours, the protein solution was passed through a 5 ml cobalt column, which was pre-equilibrated with dialysis buffer, and the recombinant protein lacking the His-tag was collected from the flowthrough.

### Inhibitor Binding Using Protein Thermal Shift

Changes in BD thermal stability due to binding of SF2523 or the MDP series molecules to the BDs employed a Protein Thermal Shift (PTS) assay. Each reaction used 20 μM concentration of recombinant BD protein in a 50 mM Tris pH 7.5 buffer containing 100 mM NaCl, 0.3 mM tris(2-carboxyethyl) phosphine HCl, and 1% dimethyl sulfoxide. Protein Thermal Shift Dye (ThermoFisher) was also added at 1× concentration. Under these conditions, 3 protein melting curves with each respective BD and 100 μM final concentration of ligand were performed from 25 to 95° C. with a ramp rate of 0.5° C./minute. Evaluation of the derivative of the fluorescence signal employed the Applied Bioscience Protein Thermal Shift Software version 1.3 to determine the average melting temperature (Tm) for each sample and the standard deviation(s) of those Tm values.

### Protein Crystallization and X-Ray Diffraction Experiments

The protein-inhibitor complexes of BRD2-BD1/MDP5, BRD2-BD2/MDP5, BRD2-BD2/SF2523, and BRD4-BD2/SF2523 were prepared by mixing each protein with the compound separately. Each mixture consisted of 5 mg/mL of protein with respective compounds to maintain a 1:10 protein: inhibitor stoichiometric ratio. All complexes were incubated on ice for 4 hours prior to crystallization. The hanging drop vapor diffusion method was used for crystallization, and drops were prepared by mixing 2 μL of reservoir well solution with 2 μL of the protein-inhibitor complexes. The 100 μL well solution of BRD2-BD1/inhibitor complex consisted of 0.2 M sodium tartrate dibasic dihydrate pH 7.5 and 20% w/v PEG 1000. The 0.1 M Bis-Tris pH 6.5, 16% w/v PEG 3350, and 0.1 M HEPES pH 7.5, 28% w/v PEG 3350 well solutions were used for the BRD2-BD2/inhibitor and BRD4-BD2/inhibitor complexes, respectively. The resulting crystals were observed after four days. For cryoprotection, 2 μL of 50% w/v PEG 3350 was added to the crystallization drop. The cryoprotected crystals were harvested and flash-cooled in liquid nitrogen to perform X-ray diffraction experiments. The ligand-free BRD4-BD2 crystals were prepared by mixing 2 μL of 5 mg/mL protein and 2 μL of well solution (0.1 M HEPES pH 7.5, 28% w/v PEG 3350). The ligand-free BRD4-BD2 crystals were cryoprotected and flashed-cooled according to the procedure described above. The X-ray diffraction experiments were performed using the LS-CAT beamline at the Advanced Photon Source of Argonne National Labs, IL. HKL2000 (Otwinowski, W. M., Processing of X-ray diffraction data collected in oscillation mode, in: Methods in Enzymology, Academic Press, 1997, pp. 307-326) was used to index, integrate, and scale the collected data corresponding to each protein/inhibitor complex. Molecular replacement used the previously published BRD2-BD2/HWV complex structure (RCSB accession number 6E6J) (Faivre, et al. (2020) Nature 578:306-310) and BRD4-BD2/89J complex structure (RCSB accession number 5UF0) (Wang, et al. (2017) J. Med. Chem., 60:3828-3850) as the search models for phasing the BRD2-BD2/MDP5, SF2523, and BRD4-BD2/SF2523 and ligand-free BRD4-BD2, respectively, using Phaser (McCoy, et al. (2007) J. Applied Crystallography 40: 658-674) in PHENIX (Adams, et al. (2010) Acta Crystallographica Section D, 66:213-221). All refinements were performed using PHENIX. COOT (Emsley, et al. (2004) Acta Crystallographica Section D, 60:2126-2132) was used for visualization and manual refinement of the structures. Structure validation was performed in PHENIX using MolProbity (Williams, et al. (2018) Protein Sci., 27:293-315) and by RCSB following submission of coordinates and phases.

### Cell Viability and Colony Formation Assay

For cell viability assay, DAOY cells from SHH-MB group and HD-MB03 from Group 3 MB were exposed to new analogs in a range of concentrations. In brief, cells were seeded in 96-well plate with a density of 3×10³ cells/well density for 16 hours. Cells were then treated with different drug concentrations for 48-72 hours. MTT assay reagent was added to the cells and after incubation for 4 hours, formazan crystals were dissolved in 200 μL DMSO, and the absorbance was determined using a microplate reader (Molecular Devices Me5). The IC₅₀ was determined by nonlinear least square regression by performing each experiment in triplicate.

Approximately 500 MB cells were seeded overnight in 6 well plates and treated with MDP5 at IC₅₀ for the colony formation assay. After 48 hours of incubation, the media was replaced by fresh media and allowed cells to grow for 2 weeks. Then, cells were fixed into 10% formalin and stained with a 0.5% solution of crystal violet dye in 20% methanol. After taking pictures, the colonies were dissolved in 10% acetic acid solution to measure the optical density.

### Effect of MDP5 on Colony Formation, Cell Cycle and Apoptosis

Colony formation ability, cell cycle analysis, and cell apoptosis assay were performed in MB cells in presence of drugs as described (Kumar et al. (2021) Biomaterials 278:121138). For the colony formation assay, after taking images, the colonies were dissolved in 10% acetic acid solution to measure the optical density (OD).

Cell cycle distribution was determined in proliferating MB cells. Cells (0.2×10⁶) were seeded in a 6 wells plate, and growth inhibition was induced by adding drug solutions at their IC₅₀ for 48 hours. Cells were harvested with 0.25% EDTA-free trypsin (Gibco) and washed twice in ice-cold PBS. Control and treated cells (0.1×10⁶) were stained with FxCycle™ PI/RNase Staining Solution (ThermoFisher Scientific) and analyzed for DNA content via flow cytometry.

For apoptosis assay, the cells were collected as above and stained with 500 μL 1× binding buffer containing 5 μL annexin V-FITC and 10 μL propidium iodide (PI) in the dark for 5 minutes. Flow cytometry (FACSCalibur™ cytometer, BD Biosciences) was performed to quantify and calculate the cell apoptosis rate. AV-FITC values were set horizontally, and PI values were set as vertical axes. All experiments were performed in triplicate.

### RT-qPCR and Western Blot Analysis

Whole RNAs in cells after treatments were extracted using an RNA extraction kit (RNAeasy, Qiagen) to detect mRNA expression. cDNA templates were synthesized by reverse transcribing the RNA using a High-Capacity RNA-to-cDNA™ Kit (Thermo Fisher). The final RT-qPCR reaction mixture was used consisting of a total volume 10 μl containing 4 μl SYBR Green master mix, 0.5 μl of each primer (10 μM) (Table 1), 2 μl of the cDNA template, and 3.5 μl RNase-free H₂O. The relative expression level of the targeted gene was measured with Lightcycler 480 (Roche). The gene expression levels were calculated according to the 2^{-ΔΔCq} method using β-actin as the internal reference gene. Each assay was repeated≥3 times.

TABLE 1
Sequences of primers used.

| Gene | Forward | Reverse |
|---|---|---|
| MYCN | CCTTGAGCGACTCAGATGATGA | GGACGCACAGTGATCGTGA |
| | (SEQ ID NO: 1) | (SEQ ID NO: 2) |
| BCL2 | GAACTGGGGGAGGATTGTGG | GCATGCTGGGGCCATATAGT |
| | (SEQ ID NO: 3) | (SEQ ID NO: 4) |
| B-actin | TTAGTTGCGTTACACCCTTTCTTG | CTGTCACCTTCACCGTTCCAGTTT |
| | (SEQ ID NO: 5) | (SEQ ID NO: 6) |

The whole protein from each treated group was extracted using RIPA buffer containing a cocktail of protease inhibitors (Sigma-Aldrich) for protein level determination. Protein concentration was determined using a BCA assay (Peirce). Next, 40 μg protein was subject to SDS-PAGE in a 10% gel. Following the transfer of targeted proteins onto polyvinylidene difluoride sheets, the membranes were washed with Tris-buffered saline plus Tween® 20 (TTBS; cat. no. WLA025; Wanleibio Co., Ltd.) for 5 minutes and then blocked with skimmed milk powder solution for 1 hour. Primary antibodies against Gli1 (1:1000), Gli2 (1:1000), MYCN (1:1,000), p-MYCN (1:1,000), Bcl-2 (1:1000), Bax (1:1000), p-PI3K (1:500), PI3K (1:1000), p-Akt (1:500), Akt (1:1000), Cyclin D1 (1:1000), and β-actin (1:1,000) were added and the membranes were incubated at 4° C. overnight. After an additional four washes with TBST, secondary IgG antibodies conjugated to horseradish peroxidase (1:5,000) were incubated with the membranes for 45 minutes at 37° C. Following a further six washes with TBST, the blots were developed using ECL start Western blotting detection kit (GE Healthcare) and the results were recorded in the iBright™ FL100 (Invitrogen). The relative expression levels of these proteins in different groups were calculated with the iBright™ software.

### Tumor Spheroid Assay

Tumor spheroids were formed using MB cells following the method described (Bhattarai, et al. (2021) J. Control. Release 329:585-597). For tumor spheroids assay, 1500 and 500 DAOY and HD-MB03 cells, respectively, were suspended in 100 μL media and seeded in ultra-low attachment (ULA) 96-well plate for a week and 50 μL of media was replaced by fresh media every third day and spheroid size was tracked under fluorescent microscope. When the tumor spheroid size reached 200 μm, spheroids were treated with SF2523, MDP1, and MDP5 at a concentration of 5.0 μM. After 7 days of the treatment period, spheroids were stained with Calcein AM (2 μM) and ethidium homodimer-1 (3 μM) (LIVE/DEAD™; L3224) to check the live/dead cells. Images were taken by confocal laser microscope (Zesis LSM 710) with 10× magnification at 40 μm depth.

### Effect of Drug Treatment on Tumor Propagating Cells and Side Population

The tumor propagating cell population in ONS-76 cells was identified based on CD15+ staining by fluorescence-activated cell sorting (FACS) analysis. Briefly, ONS-76 cells were treated with SF2523, MDP1, and MDP5 at 5.0 μM concentrations for 48 hours. Then, cells were trypsinized and 1×10⁶ cells stained with a 5 μL aliquot of APC-CD15 antibody (BioLegend; 301,908) and analyzed with flow cytometry (BD, LSR Fortessa).

For the side population (SP) assay, ONS-76 cells were preincubated with SF2523, MDP1, and MDP5 for 0.5 hour at RT. After washing thrice with staining buffer, 1×10⁶ cells were stained with 5 μg/mL of Hoechst 33342 (Thermo Scientific; 62,249) for 1 hour in an incubator with occasional shaking every 20 minutes. The PI solution 5 μL was added to exclude dead cells and analyzed with flow cytometry (LSRII, BD Biosciences).

### Animal Studies

All the animal-related experiments were performed according to the Institutional Animal Care and Use Committee (IACUC) of the University of Nebraska Medical Center's approved protocol and met federal guidelines. Subcutaneous and orthotopic xenograft models were generated in this study. For establishing the xenograft model, DAOY cells (1×10⁶) were injected into the right flank of 8-10 weeks old male and female mice. Tumor growth was measured non-invasively by using vernier calipers. For both measurements, tumor volumes were calculated by using the following formula: Volume=(Length×Width²)/2.

For the orthotopic MB model, stable luciferase-expressing DAOY cells (0.1×10⁶) were used to generate the orthotopic MB tumor in NSG mice (Kumar, et al. (2021) Biomaterials 278:121138). Bioluminescence imaging using IVIS (Spectrum) was done to monitor the tumor growth weekly after implantation surgery. The orthotopic model gradually increased in estimated tumor volumes over 6 weeks, while the subcutaneous model showed a sharp and rapid increase in tumor size starting at the 4th-week.

### In Vivo Anti-Tumor Effect

After establishing an orthotopic MB tumor, mice were randomly divided into the following three groups (n=5): i) MB bearing mice no treatment (control); ii) MB bearing mice treated i.v., with SF2523 solution (propylene glycol (20% v/v)+Cremophor® EL (30% v/v)+5% dextrose solution (50% v/v), and iii) MB bearing mice treated i.v., with MDP5 solution at the dose of 20 mg/kg every third day. A total of seven injections were given, and the mouse body weights were measured before every injection. The tumor growth was monitored by calipers in the case of the xenograft model and by IVIS imaging for the orthotopic model. For histological examination, the major organs of representative mice from each animal group were dissected and stained with H&E. Tissues after being fixed with 10% paraformaldehyde solution for 24 hours were embedded in 5 μm thick sections of paraffin, and were stained for different proteins. Stained slides were imaged at 40× magnification using an iScan HT Slide Scanner (Ventana Medical Systems, Inc, AZ).

### Statistical Analysis

The student's t-test was used to compare two different groups, and one-way ANOVA was performed for three or more groups. The data obtained were shown as the mean±S.D., and p<0.05 was considered statistically significant.

### Results

### Binding of MDP Series to BRD2 and BRD4-BD2

Structural analogs of SF2523 termed as a MDP series were synthesized (FIG. 1A). The most potent molecule MDP5 was also characterized and confirmed with ¹H NMR and mass spectrometry. A Protein Thermal Shift (PTS) assay was employed to evaluate the binding of the MDP series and SF2523 to BRD2-BD2 and BRD4-BD2. These results illustrate that MDP1 through MDP4 do not significantly enhance the thermal stability of BD2 as the change in BRD2-BD2 melting temperature (Tm) in the presence of these compounds resulted in Tm changes between −0.2° C. and 0.4° C. The BRD4-BD2 experiments showed a Tm decrease of 1.3° C. with MDP1 and MDP3. A decrease in Tm of 0.6° C. was observed for MDP2 and MDP4. In contrast, MDP5 and SF2523 increased the BRD2-BD2 Tm by 2.4° C. and 6.3° C., respectively. The Tm for BRD4-BD2 with MDP5 and SF2523 increased by 1.7 and 3.4° C., respectively. These data indicate that MDP5 represents the best binder of the MDP series and is comparable to SF2523 in terms of thermal stabilization of the BD2s.

### Verification of the Docking Method

Docking studies of SF2523, ABBV_744, and IBET_762, inhibitors with reported binding affinity Kd values, were carried out against three model proteins, RD0 (PI3Kα), BD1, and BD2. Results show that the mean errors between predicted and observed binding affinity, as measured by ΔΔG_BD1 and ΔΔG_BD2, were −1.73 and −0.46 for BD1 and BD2, respectively. These minor errors indicate that the Glide Dock program can be used to predict the binding affinity. The negative mean errors indicate that the Glide Dock scores slightly underestimated the observed binding affinity in these model proteins. The IC₅₀ of SF2523 against PI3Kα was reported to be 16 nM, using the above conversion factor, −10.63 kcal/mol. The mean error between the predicted docking score and the observed ΔG is −2.27 kcal/mol, indicating a reasonably good prediction in the protein PI3Kα (RD0 model).

### Docking Analysis of Synthesized Compounds Against Model Proteins BD1, BD2, and PI3K

To gain detailed insight into the binding interactions of the analogs with PI3Kα and BRD4 first and second bromodomain (BD1 and BD2 proteins), molecular docking studies using the Glide docking approach were performed. The docking analysis predicts that quercetin and MDP1 may have the best activities against PI3Kα, followed by MDP3 and MDP5. MDP4 may have similar activity as LY294002, a known PI3Kα inhibitor. The docking studies also indicate that LY294002 and quercetin may be dual inhibitors of PI3Kα and BD1 and BD2. Similarly, the docking studies also predict MDP1 and MDP5 to be dual inhibitors of PI3Kα and BD1 and BD2, whereas MDP3 may be a weak inhibitor of BD2 but show more selectivity toward BD1. Therefore, MDP2 and MDP3 may be more selective toward BD1. Overall, MDP1 shows the best binding toward PI3Kα, MDP5 shows the best binding toward BD1 and BD2, and MDP2 and MDP3 show more selectivity toward BD1 over BD2.

Crystal Structures Show MDP5 and SF2523 have Different Binding Modes BRD2-BD2/MDP5, BRD2-BD2/SF2523, and BRD4-BD2/SF2523 Complexes

The protein-inhibitor interactions were characterized by determining the X-ray crystal structures of ligand free BRD4-BD2, BRD2-BD2/MDP5, BRD2-BD2/SF2523, BRD2-BD1-MDP5, and BRD4-BD2/SF2523 complexes, which were resolved to resolutions of 1.22 Å, 1.20 Å, 1.27 Å, 2.50 Å, and 2.08 Å, respectively. In all cases, different density for the respective ligand was observed in the acetyl-lysine binding site.

In all inhibitor bound structures, the carbonyl oxygen of the 4H-pyran-4-one ring forms a hydrogen-bonded interaction with ND2 of Asn 429/433 (BRD2-BD2:429 and BRD4-BD2: 433) and a water-mediated hydrogen-bonded interaction with Tyr 386/390 (BRD2-BD2:386 and BRD4-BD2 390). Those two interactions were consistent with the acetyl-lysine and inhibitor-bound BRD2-BD2/BRD4-BD2 crystal structures (Romero, et al. (2016) J. Med. Chem., 59:1271-1298; Boyson, et al. (2021) Cancers 13:3606; Li, et al. (2021) J. Enzyme Inhibit. Med. Chem., 36:903-913; Sheppard, et al. (2020) J. Med. Chem., 63:5585-5623; Runcie, et al. (2018) Chem. Sci., 9:2452-2468.). Furthermore, the NE2 nitrogen atom of His 433/437 (BRD2-BD2:433 and BRD4-BD2 437) forms a water-mediated hydrogen-bonded interaction with the oxygen atom of the morpholine moiety, which is observed in all three structures described here although the bonding lengths vary. For example, 3.1 Å in BRD2-BD2/MDP5, 2.5 Å in BRD2-BD2/SF2523, and 3.5 Å in BRD4-BD2/SF2523 complexes, respectively.

Although the polar interactions are important for inhibitor recognition and specificity of binding, the acetyl-lysine binding pocket is predominantly hydrophobic and the interactions of the inhibitors reflect the same. The BRD2-BD2/MDP5 and BRD2-BD2/SF2523 complex structures illustrate that the side chains of Val 376, Leu 383, and Val 435 form van der Waals interactions with the chromone moiety of MPD5 and thienopyranone moiety of SF2523. Similar interactions are formed by Val 380, Leu 387, and Val 439 sidechains with the thienopyranone moiety of SF2523 in the BRD4-BD2/SF2523 complex. When considering the benzodioxane moiety of MDP5 and SF2523, the benzene ring forms π-π interactions with the side chain of Trp 370/374 (BRD2-BD2:370 and BRD4-BD2 374) and van der Waals interactions with the side chains of Leu 381/Leu 385 (BRD2-BD2:381 and BRD4-BD2 385. It is clear from the superimposed structures of BRD2-BD2/MDP5 and BRD4-BD2/SF2523 complexes that each compound possesses a different binding mode for the respective benzodioxane moieties.

When comparing the chromone moiety of MDPS and thienopyranone of SF2523, the 4H-pyran-4-one ring is common, but the ring fused to the pyranone differs in that SF2523 contains a five-membered thiophene ring instead of the six-membered benzene ring in MDPS. As a result of the geometric difference between the five- and six-membered rings, the relative orientation of the bond connecting to the benzodioxane moiety of SF2523 versus that of MDPS differs. Due to this slight orientational change of the bond and a 186.4° rotation of the dihedral angle, the benzodioxane moiety of SF2523 is positioned closer to the ZA loop of the acetyllysine binding pocket. The different binding mode significantly alters the interactions formed between the dioxane moiety and the protein. Specifically, the SF2523 complex structures show the benzodioxane moiety forming two water-mediated hydrogen-bonded interactions with the carbonyl oxygen of Pro 375/379(BRD2-BD2:375 and BRD4-BD2:379) and the backbone nitrogen atom of Asp 377/381(BRD2-BD2:377 and BRD4-BD2:381). In contrast, the benzodioxane moiety of MDP5 in the BRD2-BD2/MDP5 complex structure does not form any interactions with those same protein backbone atoms. Indeed, the benzodioxane moiety of MDP5 interacts directly with the side chain of Trp370.

Furthermore, the acetyl-lysine binding pockets of the BRD4-BD2 and BRD4-BD2/SF2523 structures were superimposed to identify any protein structural changes resulting from the binding of SF2523. This comparison highlighted the restructuring of the ZA loop in the BRD4-BD2/SF2523 complex structure. The side chain of Leu 385 has changed from an open conformation in the ligand-free BRD4-BD2 structure to a closed conformation in the BRD4-BD2/SF2523 structure. This structural difference was observed in both molecules in the asymmetric unit of BRD4-BD2/SF2523 complex structure, so it is unlikely to represent a crystallographic artifact. Also, when comparing molecule B of BRD4-BD2/SF2523 and the ligand-free BRD4-BD2 structures, it is clear that the ZA loop region around LEU 385 of the BRD4-BD2/SF2523 complex structure has rearranged to form a single-turn α-helix. The superimposed structures further indicate a dihedral rotation in the Leu 387 of BRD4-BD2/SF2523 complex structure. The χ1 dihedral angle (between Cα and Cβ atoms) differs by 22.3° for residue Leu 387 in the BRD4-BD2 and BRD4-BD2/SF2523 structures. All the observed changes in this region indicate BRD4-BD2 ligand recognition differs from the other BDs in BRD2 and BRD4, which do not exhibit a similar conformational change upon ligand binding. This difference may play a vital role in developing potent inhibitors that bind selectively to the acetyl-lysine binding site of BRD4-BD2.

To allow further understanding regarding inhibitor selectivity, the MDP5 and SF2523 complex structures were compared to that of SRX3212, which is a derivative of SF2523. Binding of SF2523 and SRX3212 was tested against BRD4-BD1 and BRD4-BD2 using a peptide-displacement assay (Vann, et al. (2020) Sci. Repts., 10:12027-12027). SRX3212 exhibited a 65-fold and 48-fold better binding affinity than SF2523 for BRD4-BD1 and BRD4-BD2, respectively (Vann, et al. (2020) Sci. Repts., 10:12027-12027). The BRD4-BD2/SF2523 and BRD2-BD2/SF2523 complex structures were superimposed with BRD4-BD1/SRX3212 (PDB ID:6X7C) to gain insight regarding the difference in potency of these two compounds. The SRX3212 contains an additional pyrid-3-yl-methylaminocarbonyl group attached to the benzodioxane moiety but this extension does not appear to form any additional interactions with BD1. Indeed, the only additional interaction is an intramolecular hydrogen-bond between the nitrogen of the SRX3212 amide linker with an oxygen atom of the benzodioxane. This may lower rotational entropy of SRX3212 that somehow stabilizes the binding of SRX3212 within the acetyl-lysine binding pocket. Furthermore, the superimposed BRD4-BD2/SF2523, BRD2-BD2/SF2523, and BRD4-BD1/SRX3212 structures indicate the water-mediated hydrogen bonded interaction of oxygen atoms in benzodioxane are consistent in all three structures indicating that protein desolvation upon ligand binding is unlikely to account for the affinity difference. One final difference in the BRD4-BD1/SRX3212 complex structure is the presence of a water-mediated hydrogen bonded interaction between the oxygen atom of the morpholine moiety and the side chain of Asp 141 with hydrogen bond lengths of 3.3 and 3.7 Å. While the SF2523 bound BRD2-BD2 and BRD4-BD2 structures also exhibit a water-mediated interaction between the oxygen of the morpholine moiety and the NE2 of the His side chain, these hydrogen bonds are only 2.5 and 3.1 Å in size.

When comparing the morpholine moieties of both complexes, the BRD2-BD2/MDP5 complex oxygen atom of morpholine moiety forms a water-mediated hydrogen bonded interaction with the side chain His 433. The hydrogen bonding distance between the water molecule to Asp 160, and Asp sidechain's orientation illustrate that the above-mentioned water-mediated hydrogen bonded interaction is relatively weak in the BRD2-BD1/MDP5 complex. The super-imposed structures further indicate that sequence variation, Val435 in BRD2-BD2 versus Ile162 in BRD2-BD1, could impact inhibitor affinity. The additional methyl moiety increases the surface area of Ile 162 and facilitates the formation of additional van der Waals interactions with the entire chromone moiety of MDP5 in the BRD2-BD1/MDP5 structure.

### MDP5 Inhibited MB Cell Growth by Interrupting Cell Cycle Progression and Induced Cell Apoptosis

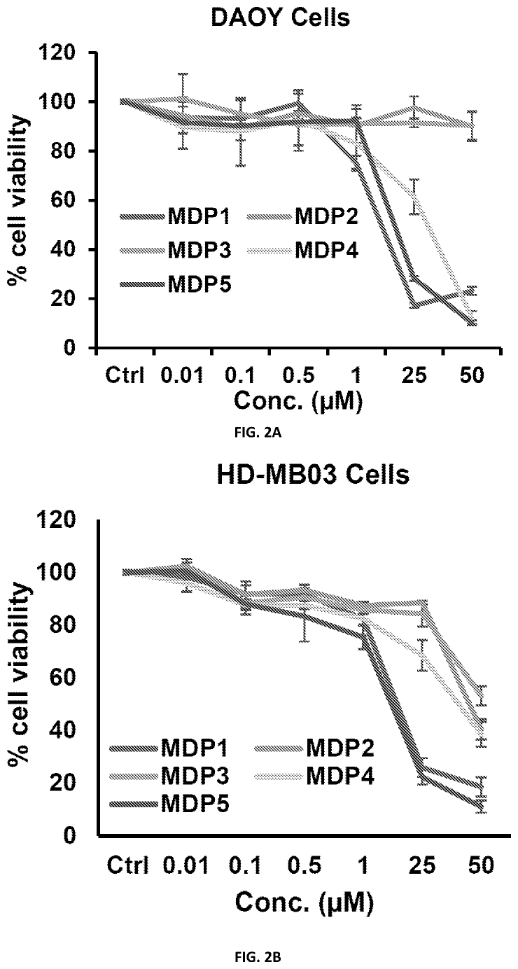

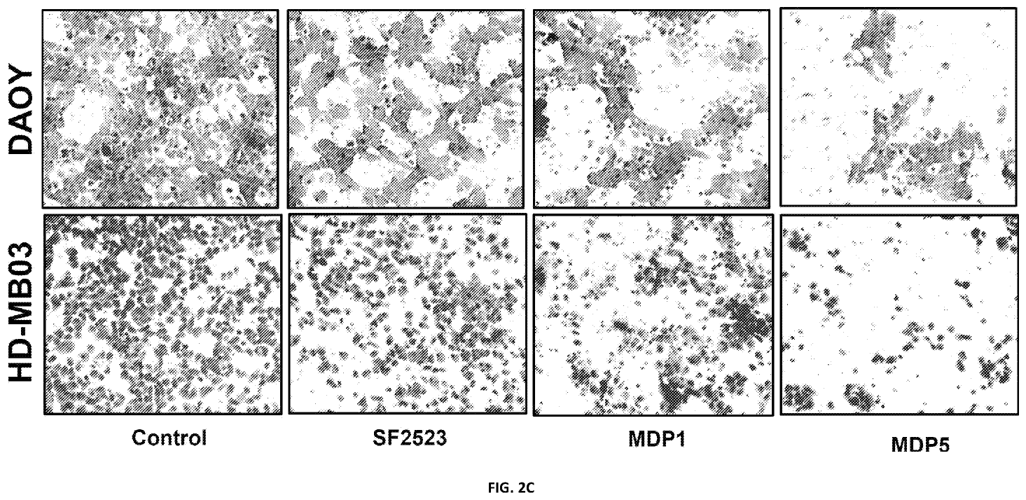

Cell proliferation was determined by MTT assay, and the results indicated that treatment with MDP5 and MDP1 significantly inhibited the proliferative ability of DAOY and HDMB-03 cell lines in a dose-dependent manner (FIGS. 2A and 2B). Among these, compounds MDP1 and MDP5 were most efficient in cell killing, wherein the IC₅₀ values in DAOY and HDMB-03 cells for MDP1 were 5.51 μM and 5.56 μM for MDP5, these were 5.57 μM in 5.14 M, respectively. In comparison, the IC₅₀ for SF2523 was 12.6 μM in DAOY and 5.14 μM in HD-MB03 cells.

Regarding the cell migration, scratch distances and width closure were measured between images from time 0 to 24 hours. After 24 hours, the untreated MB cells migrated and covered approximately 50% to 60% of the wound area quantified at time zero. However, lesser migration was observed in SF2523, MDP1, and MDP5 treated groups. Initial wound edges marked initial cell migration and were used to identify the decrease in wound width throughout the experiment. Migration distances were shown separately during the period of 0-24 hours. A significant difference in migration distance was found for the treated group compared to the control, while no difference in motility was found within the treated groups.

Further, the effect of MDPS on cell migration was investigated. The Transwell® migration assay showed that SF2523, MDP1, and MDPS decreased the amount of DAOY and HDMB-03 cells migrated from the upper surface to the lower surface of the Transwell® insert than control cells (FIGS. 2C and 2D). However, only MDPS was able to decrease migration significantly, than control in both cell lines. Therefore, these data indicate that MDPS inhibits migration and invasion of MB cells in vitro.

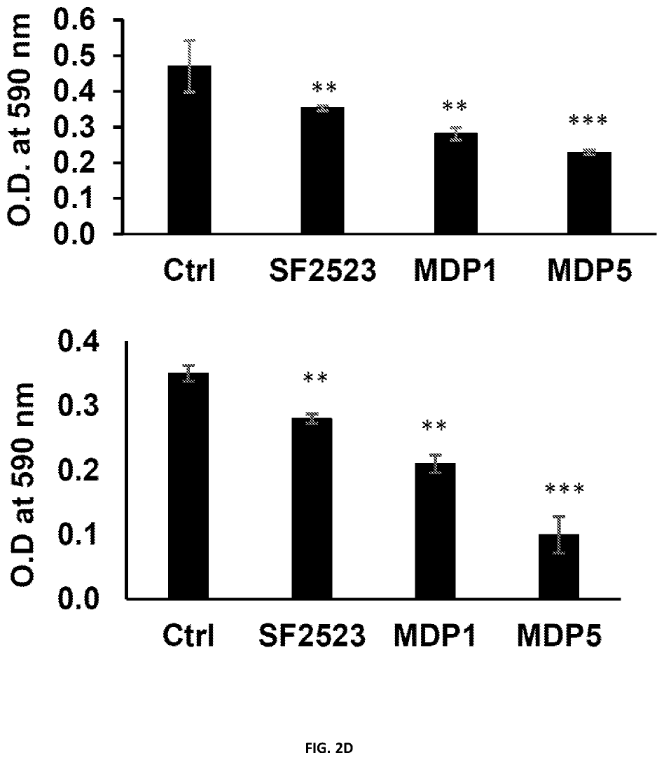

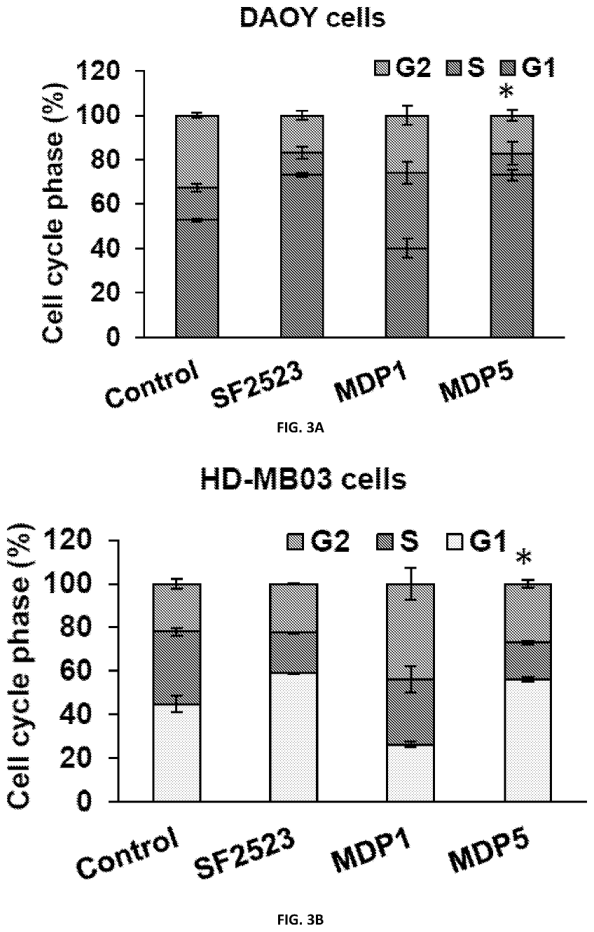

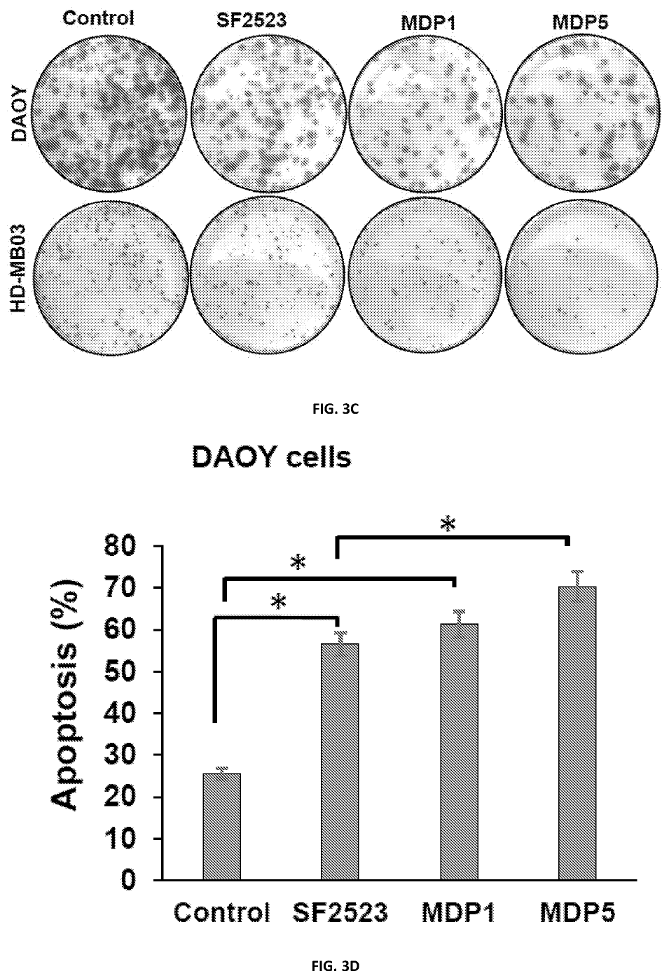

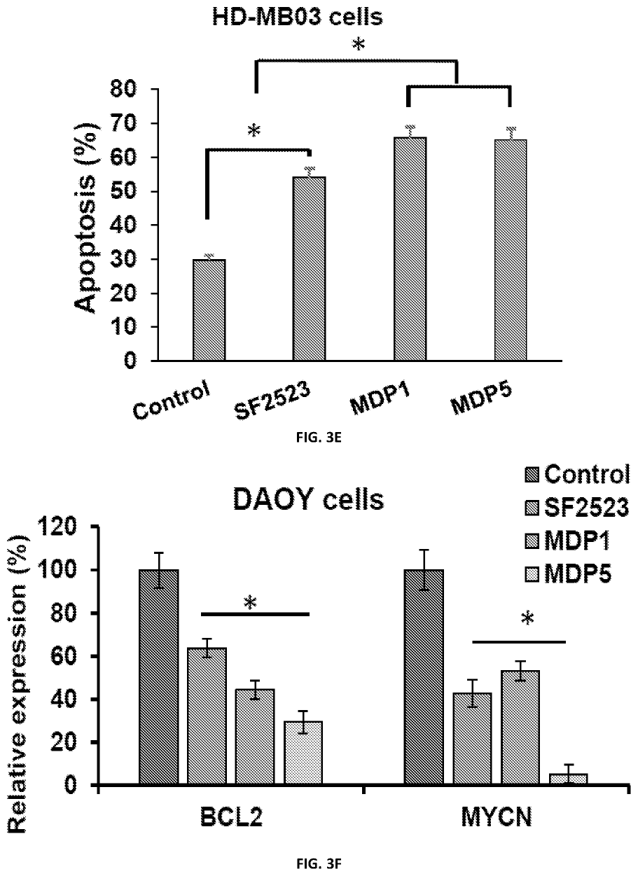

MDPS affected cell cycle progression, colony formation, and apoptosis in MB cells BRD4 has a vital role in promoting cell cycle progression from G0 to G1 and entry into the S phase in tumor cells by increasing the expression of genes such as CCND1 and CCND2 (cyclin D1 and cyclin D2), ORC2 MCM2, and PCNA (Mochizuki, et al. (2008) J. Biol. Chem., 283:9040-9048). The same trend was observed in MDPS and SF2523 treated cells, wherein these cells were arrested in the G1 phase after treatment compared to control and MDP1 (FIGS. 3A and 3B). In DAOY cells, 52.8% of cells were found in the G1 phase, whereas 73.1%, 40.1%, and 73.2% in SF2523, MDP1 and MDPS treated cells, respectively. In the case of HD-MB03 cells, 44.7% of cells were in the G1 phase, while after SF2523, MDP1 and MDP5 treatment G1 phase percentage was 58.9%, 26.2%, and 52.2%, respectively. The clonogenic assay is helpful in determining single-cell survival and its ability to multiply indefinitely to grow into a colony. In both the cell lines tested, the treatment groups showed fewer colonies than the untreated control cells at the end of 14-day incubation (FIG. 3C). However, the cells treated with MDP5 showed a significant decrease in colony formation potential as compared to SF2523 and MDP5 groups.

Further, the apoptotic assay results revealed significant BRD4 inhibition with a strong apoptosis signal in MB cells. In DAOY cells, the percentage of apoptotic cells after 48 hours of treatment was 25.61% in untreated cells, 56.50% after SF2523 treatment, 61.31% in MDP1 treated cells, and 70.35% in MDP5 treated cells (FIG. 3D). Similarly, in HD-MB03 cells percentage of apoptosis was 29.76% in untreated cells, 54% in SF2523 treated cells, 65.66% in MDP1 treated cells, and 65.15% in MDP5 treated cells (FIG. 3E). In DAOY cells, MDP5 was significantly more potent in inducing apoptosis than SF2523 and MDP1. However, in HD-MB03 cells, MDP1 and MDP5 were more potent than SF2523, but there was no significant difference within these analogs.

MDB5 Treatment Decreases Target Gene Expression at mRNA and Protein Levels

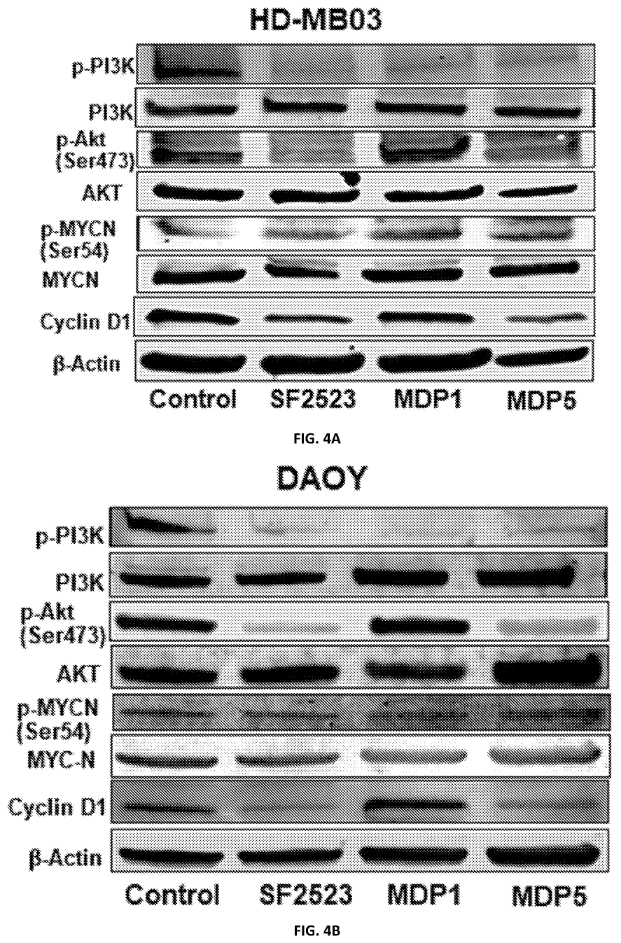

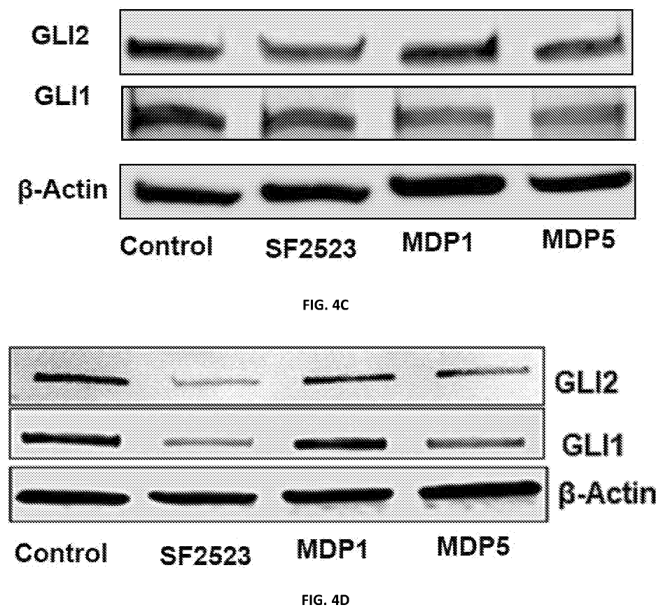

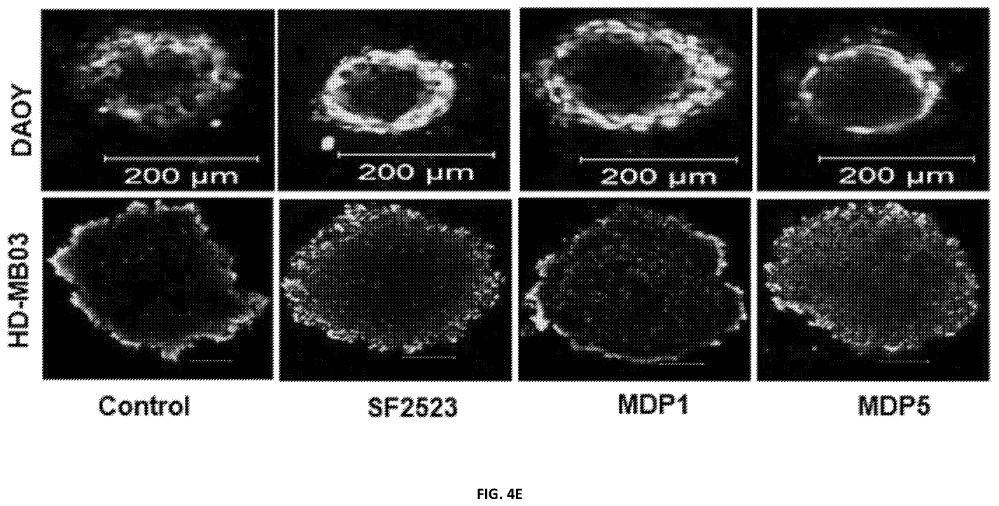

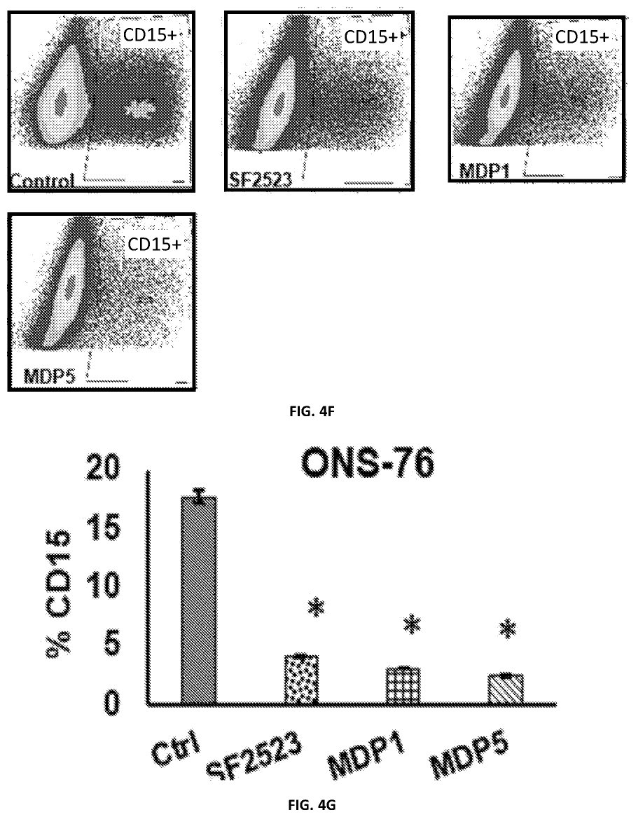

Expression of cell death related gene BCL2 was determined in MB cells and BCL2 mRNA expression was significantly reduced after MDP5 treatments (FIG. 3F). Further, cyclin D1 gene expression was also decreased significantly as compared to the control, which highlights that MDP5 was the most potent among the treatment groups. These results were confirmed by Western blot analysis. Cyclin D1, p-AKT (Ser473) and p-PI3K protein levels were reduced while p-MYCN (Ser54) level was explicitly induced in MDP5 treated cells (FIGS. 4A and 4B). The phosphorylation at Ser-54 in MYC protein is known to control its degradation, while phosphorylation at Ser473 in AKT protein is known to control its activation (Kapeli, et al. (2011) J. Biol. Chem., 286:38498-38508).

It was then tested if MDP1 and MDP5 could decrease GLI1 and GLI2 protein expression as observed following SF2523 treatment (Kumar, et al. (2021) Biomaterials 278:121138). MDP1 and MDP5 showed a decrease in levels of both proteins (FIGS. 4C and 4D). MDP5 displayed the largest reduction in GLI1 protein expression among treated HD-MB03 cells. In contrast, SF2523 exceeded all other treatment options in terms of lowering GLI2 protein accumulation.

To determine the cytotoxicity effect of these compounds in three-dimensional (3D) tumor spheroids that mimic MB tumor microenvironment, live and dead cell assays were carried out. As shown in FIG. 4E, all the treatments reduced the size of spheroids after 7 days of treatment as compared to the DMSO-treated control group. Dead cells along the margin of tumor spheroids were easily separated during staining, resulting in a tooth-shaped morphology. Cell viability of the tumor spheroids was significantly decreased following treatment with SF2523 and MDP5. The dead cells distributed across the inner core and the superficial regions of spheroids when exposed to either SF2523 or MDP5 at 5.0 μM concentration. A higher number of dead cells in the core of all the treated spheroids is possibly due to the diffusion of the drugs and high levels of cellular stress inside the 3D structure (Galateanu, et al. (2016) Int. J. Oncol., 48:2295-2302).

The proportion of CD15+ cells in various MB cells was calculated. SHH group derived DAOY cells demonstrated 3.87±0.2%, ONS-76 cells showed 23.05 f 3%, Group 3 cells HD-MB03 demonstrated 1.73±0.4%, and D283 cells demonstrated 12.97±3% CD15+ cells among them. Based on these results, ONS-76 cells were selected for further experiments. Upon treatment with MDP5, the percentage of CD15+ cell population in ONS-76 cells declined as compared to control cells (FIG. 4F). Even though the percentage of CD15+ cells was dropped significantly in all the treatment groups as compared to the control group, but no discernible difference was seen between the treated groups (FIG. 4G). It was then investigated whether treatment with SF2523 and MDP5 for 30 minutes shows a significant impact on decreasing the proportion of SP in ONS-76 cell cultures. Treatment with SF2523, MDP1, and MDP5 the percentage of SP proportion significantly decreased to 2.57%, 0.7%, and 0.9%, respectively, as compared to 4.57% for the control cells.

### MDB5 Treatment Decreases Xenograft Tumor Burden In Vivo

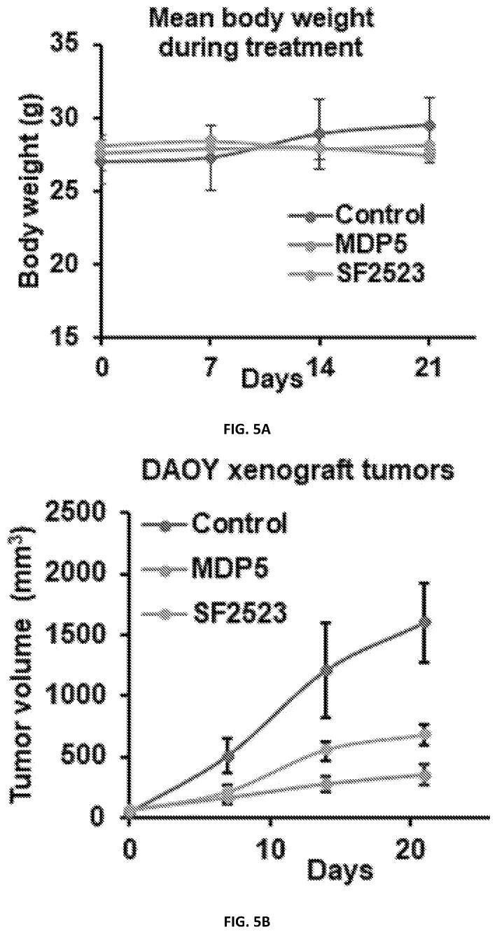

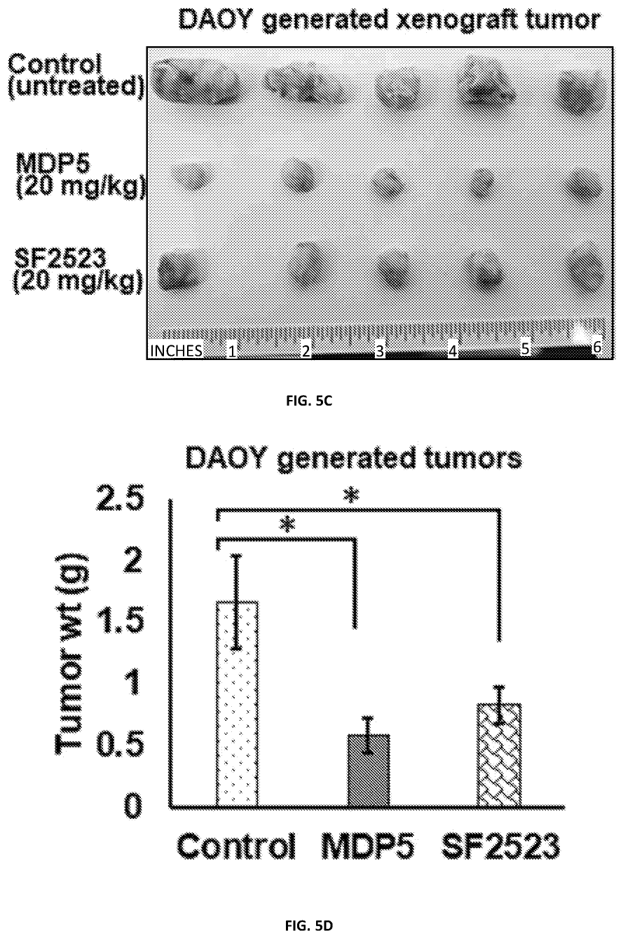

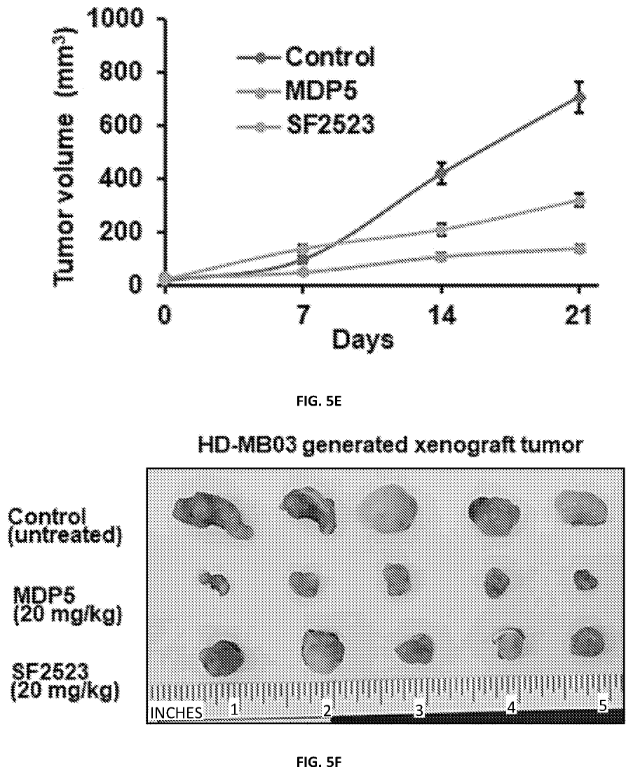

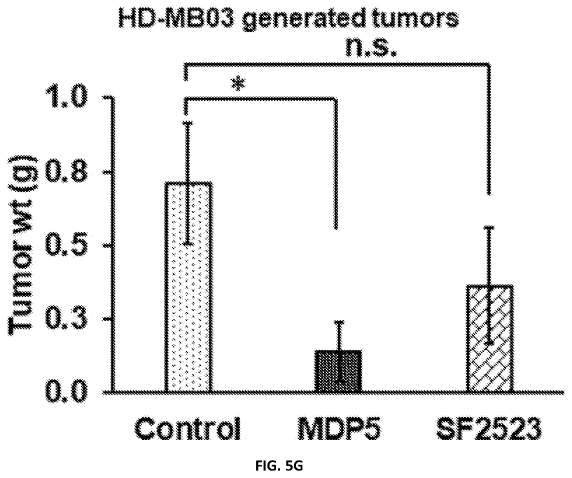

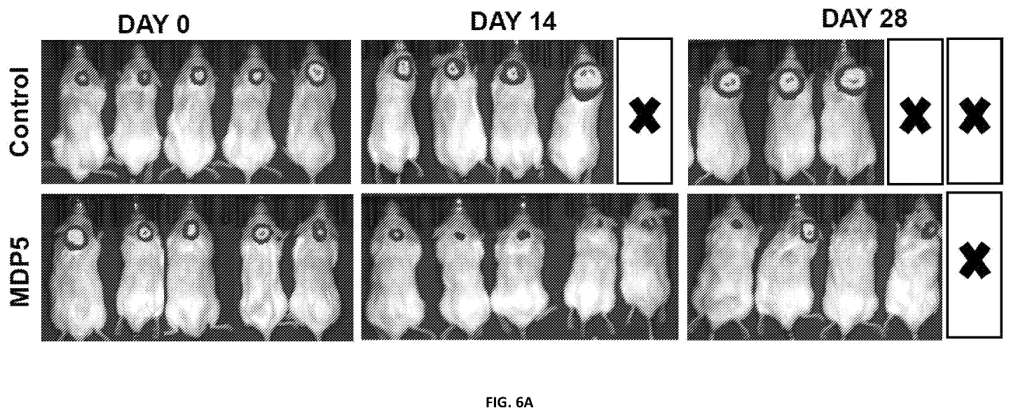

Based on in vitro results, the effect of MDP5 treatment on the inhibition of tumor growth was examined in vivo. First, a subcutaneous tumor xenograft model with DAOY and HD-MB03 cells was used (FIG. 5). At the tested dose of 20 mg/kg, animals showed no sign of toxicity, as evident by the weight curve during the treatment (FIG. 5A). Moreover, histologic examination of major organs, including liver, lung, spleen, heart, and kidneys, did not demonstrate toxicity after completion of the treatment. Tumor growth curve analysis showed that MDP5 treatment significantly affected tumor growth as compared to the vehicle group in both DAOY and HDMB-03 generated tumors. Further, H&E staining and immunohistochemical (IHC) analysis with Ki-67 showed that MDP5 treatment suppressed cell proliferation and decreased tumor burden in DAOY and HD-MB03 generated tumors. Together, these data indicate that MDP5 has potent anti-tumor activity in vivo in the xenograft model and decreased MB tumor burden and cellular proliferation without significant toxicity.

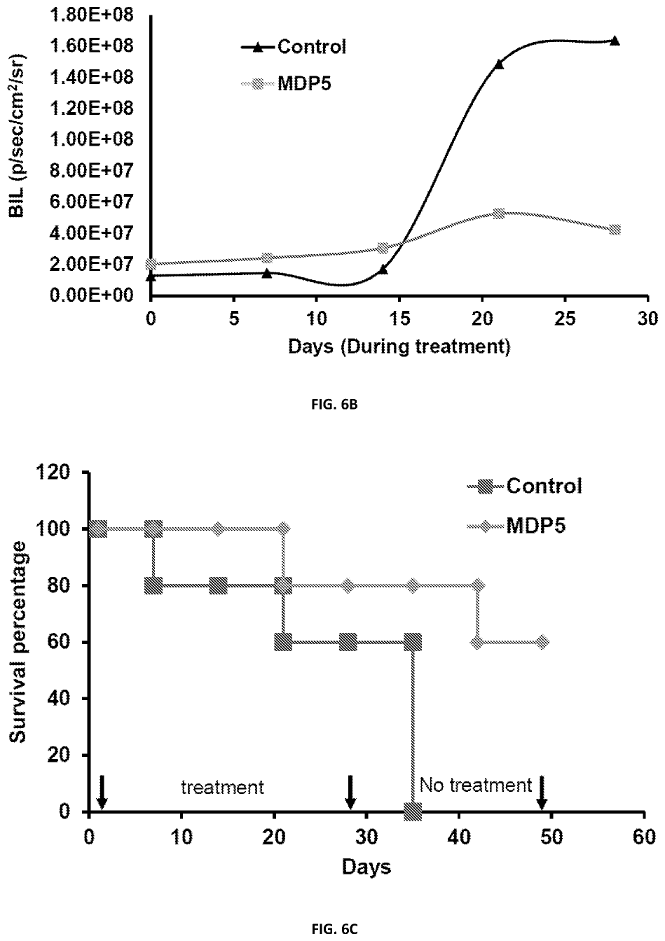

Next, it was tested whether MDP5 treatment suppresses MB growth in an intracranial orthotopic xenograft model. DAOY cells stably expressing green fluorescent protein (GFP)/luciferase were injected into the cerebella of NSG mice. After twenty-one days of implantation, the tumor signal was measured with IVIS, and animals were divided into the vehicle (propylene glycol) or MDP5 treated groups. A drug solution was injected every three days for 28 days, and bioluminescence imaging was performed once a week. IVIS imaging on day 1 (the start of injection) and on day 28 (after the last injection) of these mice from each group showed that treatment with MDP5 had a significant effect on the suppression of tumor growth in vivo (FIG. 6A). The bioluminescence (BLI) signal measurement showed a rapid increase in control animals, while a low BLI signal was detected in MDP5-treated mice (FIG. 6B).

These mice were kept without any further treatment for the survival study. When the animal started showing distress and tilting its head due to the high tumor burden, the animal was sacrificed, and its brain was harvested. As shown in FIG. 6C, in the MDP5 treatment group, four animals out of five survived up to 49 days in an orthotopic xenograft model, while the control animals were all dead by day 35.

BRD4 binds histone proteins and works as a transcriptional regulator to induce tumor growth and the inflammatory response by influencing the expression of Hh signaling pathway components and Myc (Zhou, et al. (2020) Front. Pharmacol., 11:1043). Therefore, BRD4 inhibition has been demonstrated to be a promising therapeutic approach to treat cancer. Since the first small molecular BRD4 inhibitor JQ1 was discovered, several categories of inhibitors with different chemical scaffolds have been identified. Among these reported inhibitors, I-BET762, OTX015, and CPI-0610 have already been used in the phase of clinical trials for cancer treatment. However, most of these compounds are non-specific for BET family members and cause excess side effects (Zhou, et al. (2020) Front. Pharmacol., 11:1043).

A common occurrence in MB is an upregulation of the SHH and MYC signaling pathways. The SHH upregulated MB has an intermediate prognosis, while MYC upregulated MB are very aggressive with the worst prognosis. Although these are classified as distinct groups, MYCN and MYCL1 are highly expressed in the SHH subgroup (Northcott, et al. (2012) Nature 488:49-56). Consequently, BRD4 inhibitors are quite useful from a therapeutic standpoint in the treatment of MB. However, MB treatment by BRD4 inhibition is challenging due to the development of chemoresistance by PI3K, which also plays a key role in MB cell growth, NSC proliferation, and tumorigenesis. This chemoresistance can be overcome by modulating MYC using PI3K/BRD4 dual inhibitors.

Herein, a novel BRD4/PI3K dual inhibitor MDP5, which is an SF2523 analog, was synthesized. The MDP5 and SF2523 bound X-ray crystal structures of BRD2-BD1/MDP5, BRD2-BD2/MDP5, BRD2-BD2/SF2523, and BRD4-BD2/SF2523 complexes were used for the characterization of protein-inhibitor interactions and evaluation of the structural changes occurred in BRDs upon binding of inhibitors. When comparing the crystal structures of BRD2-BD2/MDP5 and BRD2-BD2/SF2523, it indicated a small but intriguing change in the binding mode of MDP5 and SF2523 in the acetyl-lysine binding pocket. The benzodioxane moiety of SF2523 in the BRD2-BD2/SF2523 structure had moved towards the ZA loop. It forms additional water-mediated hydrogen-bonded interactions with the carbonyl oxygen of the Pro 375 and backbone nitrogen of the Asp 377 compared to MDP5. Those additional hydrogen-bonded interactions further contribute to the stabilization of SF2523 within the BRD2-BD2 acetyl-lysine binding pocket.

The observed conformational change of Leu 385 (open to closed-form), ZA loop rearrangement, and the dihedral rotation of Leu 387 upon binding of SF2523 to BRD4-BD2 may account for the PTS results. In contrast to the induced fit for ligand binding to BRD4-BD2, structural data for BRD2-BD2 indicates that the acetyl-lysine binding site does not change upon binding of ligands as observed when comparing the ligand-free BRD2-BD2 structure with the structures of the numerous BRD2-BD2/ligand complexes. Therefore, the modest thermal stabilization observed in the BRD2-BD experiments indicates that the MDP compounds are binding to BRD2-BD in a manner consistent with other known ligands but form only a minimum of interactions. In contrast, when these same MDP compounds bind to BRD4-BD, they can induce structural dynamics in the ZA loop but fail to form stabilizing interactions, which results in the 0.6 to 1.3° C. thermal destabilization of those complexes. This further indicates that MDP derivatives should account for the structural differences between the ligand-free forms of BRD2-BD2 and BRD4-BD2 and seek to bind BRD4-BD2 to prevent the conformational change. This would result in a potent inhibitor of BRD4-BD2 that can only weakly bind BRD2-BD2 due to steric hindrance with the ZA loop. BRD4 and PI3K inhibition assays confirmed its inhibitory potential and mode of action. While MDP5 (5.5 μM) showed higher potency in DAOY cells as compared to SF2523 (12.6 μM), similar potency was observed in HD-MB03 MB (MYC amplified) cells. In cell migration, apoptosis, cell cycle, and wound healing assays MDP5 showed higher potency compared to SF2523 in both DAOY and HD-MB03 cell lines. MDP5 showed a decrease in the expression of target downstream proteins like p-AKT, MYCN, Cyclin D1 with the increasing degradation of MYCN protein indicated by upregulation of p-MYCN (Ser 54). These results agree with a report where SF2523 blocks PI3K activation and down-regulates MYCN and Cyclin D1 in vivo (Andrews, et al. (2017) Proc. Natl. Acad. Sci., 114:E1072-E1080). It is well established that the SHH-GLI signaling pathway is activated by multiple non-canonical activation methods in cancers. Consequently, SMO inhibition is less effective against tumors with noncanonical SHH-GLI signaling, which jeopardizes the therapeutic effectiveness of these antagonists. The PI3K/AKT/mTOR pathway is known to upregulate GLI1/GLI2 transcription and enhance protein levels (Kenny, et al. (2022) J. Med. Chem., 65:14261-14275). Accordingly, MDP5 showed downregulation of these proteins in the results (FIGS. 4C and 4D), indicative of an effective anti-cancer therapy.

The structural results indicate that MDP5 retains much of the essential structural properties of parent compound SF2523 to support BD binding. It is clear from a comparison of the BRD4-BD1 complexes with MDP5 that it competes with acetyl-lysine binding and maintains the conserved hydrogen bond with Asn140 found in acetyl-lysine-BD complexes but does not interfere with the distinctive water shell of BD1. The morpholino ring of MDP5 is parallel to the a-helices of BD1 and buried deep in the pocket to support binding. The benzodioxane group of MDP5 faces the ZA loop in BD1, which places it at right angles to the a-helices and interacts with the hydrophobic cage formed by the side chains of Trp81, Pro82, and Leu92. The chromone component of MDP5 is stabilized by the side chains of Val87 and Ile146, while the amide nitrogen of the Asn140 side chain and the hydroxyl group of Tyr97 in BD1, interacts with the carbonyl oxygen in the chromone group by hydrogen bonding. Further, additional hydrogen bonds between Trp 81, G1n85, and Asp88 in the ZA loop of BD1 hold the dioxane ring of MDP5 in position.

Cancer stem cells, often called tumor-initiating cells, are capable of self-renewal which contributes to tumor growth and resistance. Human MB and Patched mutant mouse model cells that express the progenitor markers Math1 and CD15 are known to have the ability to proliferate and a reduced propensity to undergo apoptosis and differentiate. The results herein confirmed that BRD4 and PI3K inhibition decreased CSCs and SP cells, demonstrating that they are effective in eradicating chemo-resistant cell populations.

For efficacy in tumor xenograft and safety studies, compound MDP5 was selected in established human MB DAOY and HDMB-03 xenograft model in NSG mice. Mice bearing subcutaneous tumors treated with 15 mg/kg dose of SF2523, MDP5 and MDP1 demonstrated the significant inhibition of tumor growth as compared to vehicle treated control. It was observed that MDP5 was able to significantly reduce the tumor burden compared to other treatments. Further, at the tested dose MDP5 was well tolerated in mice as assessed by minimal changes in body weight during the course of treatment and histological examination of vital organs as compared to vehicle control.

Notably, the physiochemical properties of MDP5 are consistent with compounds which are shown to cross the blood-brain barrier (BBB). For example, the molecular mass is lower than 450 a.m.u., the compound is lipophilic with a CLogP of 2.99, has <3 hydrogen bond donors, and a topological polar surface area (tPSA) of 57.23. Each of these physicochemical parameters are known to contribute to BBB penetrability (Kenny, et al. (2022) J. Med. Chem., 65:14261-14275; Sethi, et al. (2022) J. Control. Release 350:668-687). Indeed, tPSA, which is a descriptor to define the sum of surfaces of polar atoms in a molecule, is one of the most useful parameters for predicting molecular transport properties (Xiong, et al. (2021) J. Med. Chem. 64:13152-13173). Further, the 1,4-dioxane ring fused onto the aromatic ring reduces the tPSA with respect to other analogs. Since these properties are indirect indicators of BBB penetration ability of molecules, it was tested whether MDP5 treatment suppresses MB growth in an intracranial orthotopic MB model, and it was observed that MDP5 significantly suppressed tumor growth in vivo. After the final treatment, these mice were permitted to live, and their brains were removed when they began to act listless. H&E staining and IHC analysis of Ki-67 revealed a much larger tumor at the cerebellum region of the vehicle-treated mouse as compared to MDP5 and SF2523 treated mice.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A compound of formula (I): 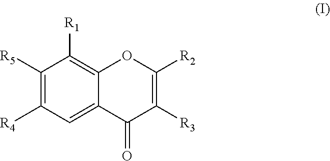
wherein:
R₁ is selected from the group consisting of halo alkyl benzodioxine, halo, alkyl benzodioxine, amino benzodioxane, H, trialkyl isoxazole, dialkylisoxazole, haloalkoxy alkylpyridine alkylbenzamide, benzodioxane alkylamine, benzodioxane, aryl, and imidazo pyridazine alkyne;
R₂ is selected from the group consisting of H, alkyl morpholine, morpholinyl, trihalophenoxy, aryl, and trihaloanisole;
R₃ is selected from the group consisting of alkyl morpholine, morpholinyl, H, and halo;
R₄ is selected from the group consisting of H, alkyl benzodioxine, alkoxy alkylbenzene, halo, trialkylisoxazole, dialkylisoxazole, imidazopyridazine, aryl, benzyloxy, and alkyl; and
R₅ is selected from the group consisting of H, benzodioxane, and aryl,
wherein any one of R₁, R₂, R₃, R₄, and R₅ may be optionally substituted,
or a pharmaceutically acceptable salt thereof.

2: The compound of claim 1, wherein:
R₁ is selected from the group consisting of optionally substituted benzodioxane, halo, dialkylisoxazole, 3, 5-dimethylisoxazol-4-yl, N-benzyl-4-fluoro-5-methoxybenzamide, and amino-1,4-benzodioxane;
R₂ is selected from the group consisting of H, halo, H, morpholinyl, and trihalophenoxy;
R₃ is selected from the group consisting of H, halo, and morpholinyl;
R₄ is selected from the group consisting of H, halo, phenyl alkoxy, an optionally substituted benzodioxane, dialkylisoxazole, 3, 5-dimethylisoxazol-4-yl, alkyl, and imidazopyridazine; and
R₅ is H or an optionally substituted benzodioxane.

3: The compound of claim 1, wherein R₁ is selected from the group consisting of N-benzyl-4-fluoro-5-methoxybenzamide; 3, 5-dimethylisoxazol-4-yl; amino-1, 4-benzodioxane; 2, 3-dihydrobenzo [b] [1,4]dioxin-6-yl; and 7-halo-2,3-dihydrobenzo [b] [1,4] dioxin-6-yl.

4: The compound of claim 1, wherein R₂ is selected from the group consisting of H, halo, morpholinyl, and 2, 4, 6-trihalophenoxy.

5: The compound of claim 1, wherein R₃ is morpholinyl.

6: The compound of claim 1, wherein R₄ is selected from the group consisting of H; halo; benzyloxy; 3, 5-dimethylisoxazol-4-yl; lower alkyl; imidazo [1,2-b] pyridazin-3-yl; 2, 3-dihydrobenzo [b] [1,4] dioxin-6-yl; and 7-halo-2, 3-dihydrobenzo [b] [1,4]dioxin-6-yl.

7: The compound of claim 1, wherein R₅ is selected from the group consisting of H; 2, 3-dihydrobenzo [b] [1,4] dioxin-6-yl; and 7-halo-2,3-dihydrobenzo [b] [1,4] dioxin-6-yl.

8: The compound of claim 1, wherein R₃, R₄, and R₅ are H.

9: The compound of claim 1, wherein R₂, R₄, and R₅ are H; R₃ is morpholinyl; and R₁ is an optionally substituted 1,4-benzodioxane.

10: The compound of claim 9, wherein R₁ is 7-halo-2, 3-dihydrobenzo [b] [1,4] dioxin-6-yl.

11: The compound of claim 1 selected from the group consisting of:

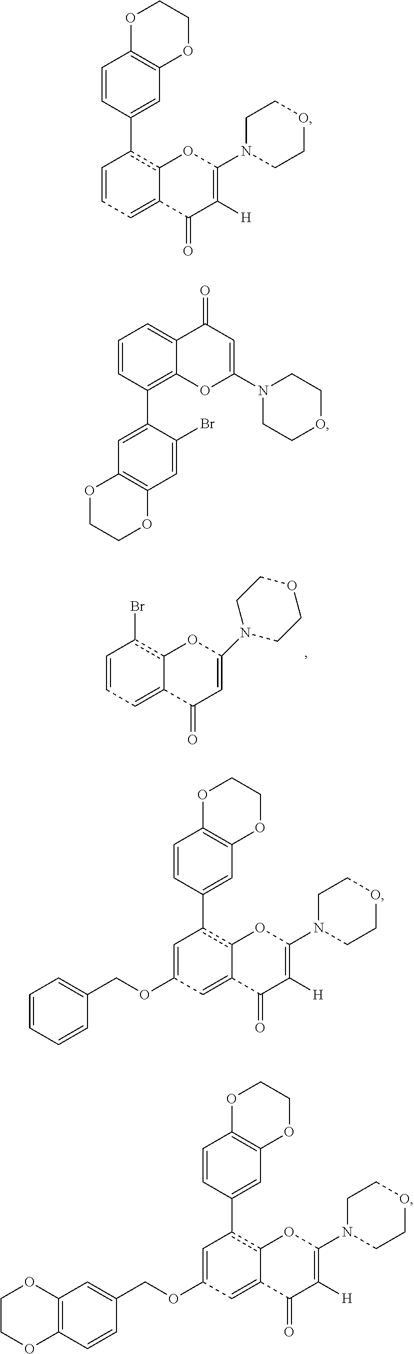

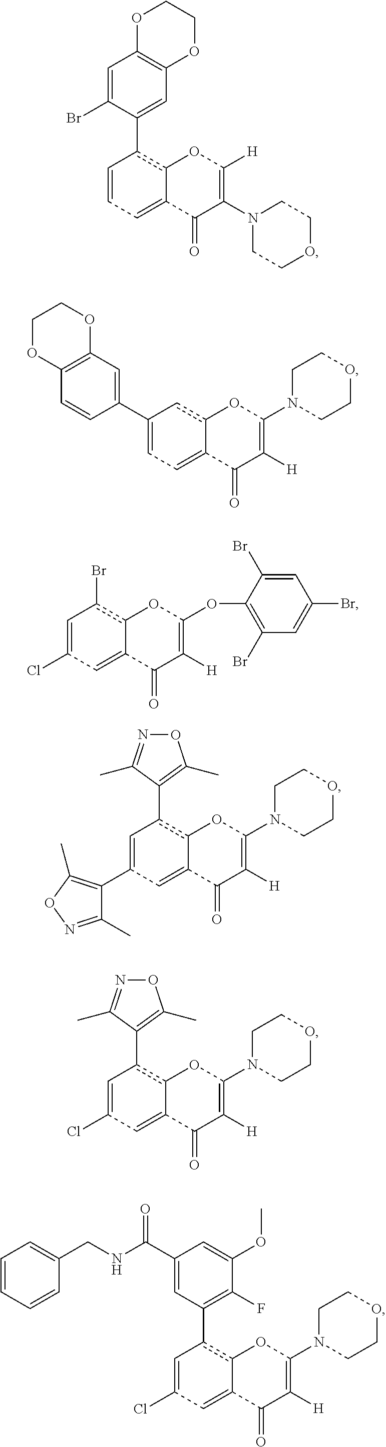

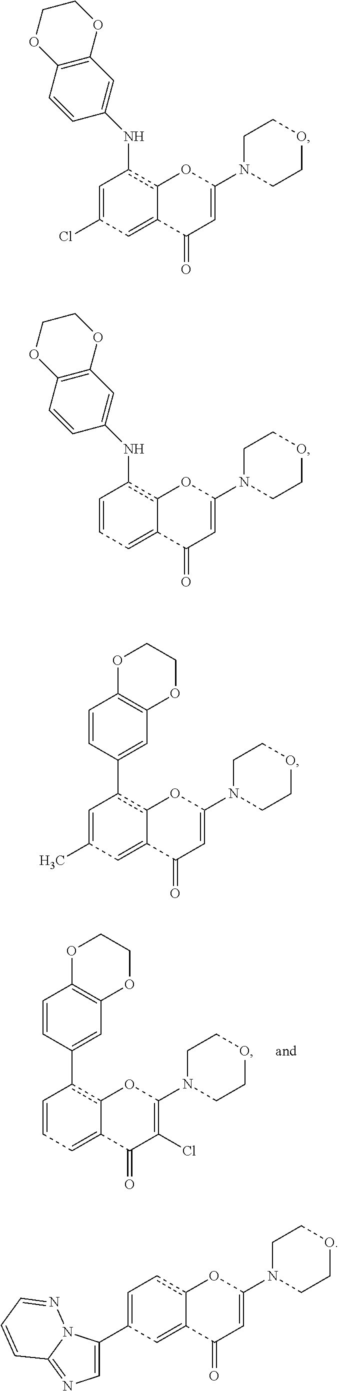

12: A compound selected from the group consisting of:

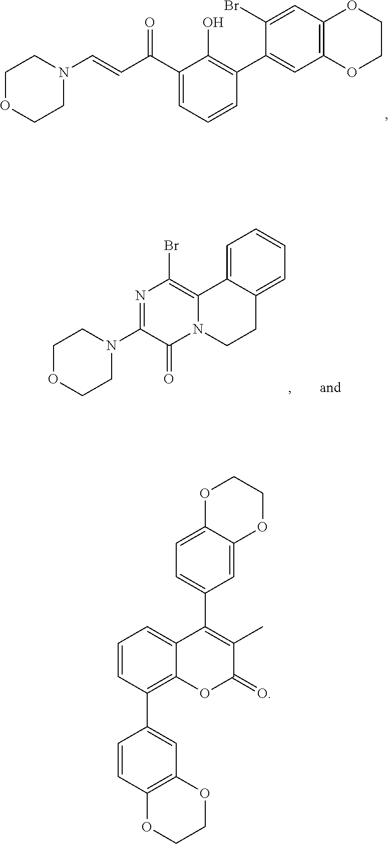 [SMILES: O=C(/C=C/N1CCOCC1)c1cccc(-c2cc3c(cc2Br)OCCO3)c1O ; O=c1c(N2CCOCC2)nc(Br)c2n1CCc1ccccc1-2 ; Cc1c(-c2ccc3c(c2)OCCO3)c2cccc(-c3ccc4c(c3)OCCO4)c2oc1=O]

13: A compound of claim 1, which is: 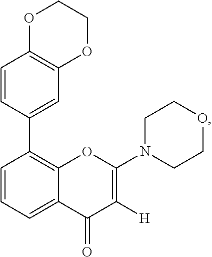 [SMILES: O=c1cc(N2CCOCC2)oc2c(-c3ccc4c(c3)OCCO4)cccc12]
or a pharmaceutically acceptable salt thereof.

14: A composition comprising a compound of claim 1 and a pharmaceutically acceptable carrier.

15: A composition comprising the compound of claim 12 and a pharmaceutically acceptable carrier.

16: A method of inhibiting bromodomain-containing protein 4 (BRD4) and phosphoinositide 3-kinase (PI3K) activity, said method comprising contacting BRD4 and PI3K with a compound of claim 1.

17: A method of inhibiting bromodomain-containing protein 4 (BRD4) and phosphoinositide 3-kinase (PI3K) activity, said method comprising contacting BRD4 and PI3K with the compound of claim 12.

18: The method of claim 16, which is in vitro.

19: The method of claim 16, wherein the method comprises contacting a solution, cell, tissue, or subject expressing BRD4 and PI3K with said compound.

20: A method of treating, inhibiting, and/or preventing a fibrotic disease or cancer in a subject in need thereof, said method comprises administering a compound of claim 1 to said subject.

21: A method of treating, inhibiting, and/or preventing a fibrotic disease or cancer in a subject in need thereof, said method comprises administering the compound of claim 12 to said subject.

22: The method of claim 20, wherein said fibrotic disease is selected from the group consisting of liver fibrosis, lung fibrosis, kidney fibrosis, and heart fibrosis.

23: The method of claim 20, wherein said cancer is selected from the group consisting of prostate cancer, bladder cancer, renal cancer, gastric cancer, liver cancer, pancreatic cancer, colorectal cancer, cancers of the central nervous system, breast cancer, melanoma, hematological cancers, lymphomas, multiple myeloma, colon cancer, thyroid cancer, lung cancer, ovarian cancer, stomach cancer, cervical cancer, testicular cancer, kidney cancer, carcinoid tumors, and bone cancer.

24: The method of claim 23, wherein said cancer is medulloblastoma.

25: The method of claim 20, further comprising administering an additional therapeutic agent to the subject.

26: The method of claim 25, wherein said additional therapeutic agent is an anti-cancer drug, anti-inflammatory drug, or immune-modulatory drug.


SEQUENCE LISTING (6 sequences)

SEQ ID NO: 1 - DNA, 22 bp, synthetic construct
  source 1..22: mol_type = other DNA
ccttgagcgactcagatgatga

SEQ ID NO: 2 - DNA, 19 bp, synthetic construct
  source 1..19: mol_type = other DNA
ggacgcacagtgatcgtga

SEQ ID NO: 3 - DNA, 20 bp, synthetic construct
  source 1..20: mol_type = other DNA
gaactgggggaggattgtgg

SEQ ID NO: 4 - DNA, 20 bp, synthetic construct
  source 1..20: mol_type = other DNA
gcatgctggggccatatagt

SEQ ID NO: 5 - DNA, 24 bp, synthetic construct
  source 1..24: mol_type = other DNA
ttagttgcgttacaccctttcttg

SEQ ID NO: 6 - DNA, 24 bp, synthetic construct
  source 1..24: mol_type = other DNA
ctgtcaccttcaccgttccagttt